(19) United States
(12) Patent Application Publication
(10) Pub. No.: US 2014/0017336 A1
(43) Pub. Date: Jan. 16, 2014

(54) CO-ADMINISTRATION OF ARSENIC COMPOUNDS AND ANTI-HERPES VIRUS ANTI-VIRALS

(71) Applicants: Mark D. Sides, New Orleans, LA (US); Joseph A. Lasky, New Orleans, LA (US)

(72) Inventors: Mark D. Sides, New Orleans, LA (US); Joseph A. Lasky, New Orleans, LA (US)

(73) Assignee: ADMINISTRATORS OF THE TULANE EDUCATIONAL FUND, New Orleans, LA (US)

(21) Appl. No.: 14/007,142

(22) PCT Filed: Apr. 6, 2012
(86) PCT No.: PCT/US12/32634

§ 371 Date: Sep. 24, 2013

Related U.S. Application Data
(60) Provisional application No. 61/516,669, filed on Apr. 6, 2011.

(51) Int. Cl. A61K 33/36; A61K 45/06; A61K 31/522

(52) U.S. Cl. CPC A61K 33/36; A61K 31/522; A61K 45/06
USPC 424/623

(57) ABSTRACT

It has now been discovered that arsenic induces herpes viruses latent in infected cells to reactivate to the lytic stage. Herpes viruses in the lytic stage activate anti-herpes virus anti [symbol] viral agents. Co-administration of arsenic compounds and anti-herpes viral agents to a population of cells infected with a herpes virus results in the death of the cells and inhibits proliferation of the virus. The invention is therefore useful for reducing a subject's population of cells infected with herpes viruses, particularly Epstein-Barr virus.

### CROSS-REFERENCE TO RELATED APPLICATIONS

This application claims the benefit of U.S. Provisional Patent Application No. 61/516,669, filed Apr. 6, 2011, which is hereby incorporated by reference.

### STATEMENT OF FEDERAL FUNDING

Not applicable.

### BACKGROUND OF THE INVENTION

Epstein-Barr virus (EBV) is a ubiquitous gamma herpes virus with an estimated 90% of the human population seropositive by adulthood. Initial infection with EBV is usually subclinical, but may manifest as infectious mononucleosis. EBV infection eventuates in viral latency and lifelong persistent infection of the host. Of clinical relevance, EBV has been implicated in a number of lymphocyte malignancies including Burkitt's lymphoma, B-cell lymphoproliferative disease, Hodgkin's disease, T-cell lymphomas, and post transplant lymphoproliferative disease (Thompson and Kurzrock, 2004, Clin Cancer Res 10:803-821). In addition, EBV infection has been linked with gastric carcinomas and anaplastic nasopharyngeal carcinoma (e.g., Nakamura et al., 1994, Cancer, 73:2239-2249; Oda et al., 1993, Am J Pathol. 143:1063-1071; Raab-Traub, 2002, Semin. Cancer Biol. 12:431-441; Shibata et al., 1991, Am J Pathol. 139:469-474; Takada, 2000, Mol. Pathol. 53:255-261; Takano et al., 1994, J. Cancer Res. Clin. Oncol. 120:303-308; Tao and Chan, 2007, Expert Rev. Mol. Med. 9:1-24; Vasef et al., 1997, Ann. Otol. Rhinol. Laryngol. 106:348-356; Wolf et al., Nat. New Biol. 244:245-247). EBV has also been implicated in the progression of idiopathic pulmonary fibrosis where detectable latent EBV proteins in lung epithelial tissue portend a poorer prognosis (Tang et al., 2003, J. Clin. 41:2633-2640).

Maintenance of viral latency is essential for host immune evasion and viral persistence. The EBV encoded Latent Membrane Protein 1 (LMP1) is required for EBV mediated immortalization of B-cells, and is a bona fide oncogene as demonstrated by its ability to transform rodent fibroblast (Eliopoulos et al., 1996, Oncogene 13:2243-2254; Moorthy and Thorley-Lawson, 1993, J. Virol. 67:2637-2645; Peng and Lundgren, 1992, Oncogene 7:1775-1782; Wang et al., 1985, Cell 43:831-840). LMP1 signaling is constitutive and has been reported to upregulate interferon stimulated genes creating an antiviral environment within the cell (Zhang et al., 2004, J. Biol. Chem. 279:46335-46342; Zhang et al., 2001, J. Virol. 75:12393-12401). Additionally, conditional expression of LMP1 in EBV infected cells has been shown to inhibit reactivation of the EBV lytic program (Adler et al., 2002, Proc. Natl. Acad. Sci. USA 99:437-442).

Current antiviral therapies targeting EBV positive cells rely on the activation of nucleoside analogue pro-drugs by a viral-specific protein kinase which is expressed only during the lytic phase (Meng et al., 2010, J. Virol. 84:4534-4542). Given that the virus is latent in the majority of EBV-positive tumors, a number of strategies have been employed to enhance lytic reactivation and induce susceptibility to pro-drug antivirals in cell culture (Daibata et al., 2005, J. Virol. 79:5875-5879; Feng et al., 2004a, J. Natl. Cancer Inst. 96:1691-1702; Feng et al., 2004b, J. Virol. 78:1893-1902; Feng et al., 2002, Cancer Res. 62:1920-1926; Feng and Kenney, 2006, Cancer Res. 66:8762-8769; Hui and Chiang, 2010, Int. J. Cancer 126:2479-2489; Jung et al., 2007, Cancer Lett. 247:77-83; Moore et al., 2001, Antimicrob. Agents Chemother. 45:2082-2091; Westphal et al., 2000, Cancer Res. 60:5781-5788).

Reactivation of the EBV lytic cycle begins with expression of the immediate early (IE) proteins Zta and Rta, which activate expression of the early proteins responsible for viral genomic replication. The EBV immediate early transactivator, Zta, has been shown to be essential and sufficient to reactivate EBV and initiate viral replication (Speck et al., 1997, Trends Microbiol. 5:399-405). Zta is expressed at a very low level during latency and mechanisms to prevent spontaneous reactivation have been proposed (Yin et al., 2004, Virology 327:134-143). Zta transactivates its own expression during reactivation and induces transcription of Rta, Zta responsive EBV early genes, and cellular genes (Flemington and Speck, 1990, J. Virol. 64:1227-1232). The EBV early protein, DNA polymerase processivity factor, is encoded by the BMRF1 gene and is essential for lytic replication. The BMRF1 gene is Zta responsive and is an indicator of lytic reactivation (Neuhierl and Delecluse, 2006, J. Virol., 80:5078-5081).

The promyelocytic leukemia protein nuclear body (PML NB) is a proteinaceous substructure that can influence a variety of nuclear processes including transcriptional regulation, telomere maintenance, and regulation of apoptosis (Block et al., 2006, Mol. Cell Biol. 26:8814-8825; Boisvert et al., 2000, J. Cell Biol. 148:283-292; Yeager et al., 1999, Cancer Res. 59:4175-4179). The PML protein is a known interferon stimulated gene (Lavau et al., 1995, Oncogene 11:871-876; Stadler et al., 1995, Oncogene 11:2565-2573), which suggests a role in modulating the host's innate cellular response to viral infection. Targeted disruption of the PML NB by herpes viruses has been observed at initiation of lytic reactivation and is consistent across the Herpesviridae family. Members of alpha (Herpes Simplex Virus 1 (HSV1)), beta (Human Cytomegalovirus (CMV)) and gamma (EBV) subfamilies encoded IE proteins (ICP0, IE1, Zta, respectively) have been shown to interact with and disperse PML NB by distinctive mechanisms (Adamson and Kenney, 2001, J. Virol. 75:2388-2399; Ahn et al., 1998, Mol. Cell Biol. 18:4899-4913; Bowling and Adamson, 2006, Virus Res. 117:244-253; Everett and Maul, 1994, EMBO J. 13:5062-5069; Kelly et al., 1995, J. Gen. Virol. 76 (Pt 11):2887-2893; Maul and Everett, 1994, J. Gen. Virol. 75 (Pt 6):1223-1233). Moreover, at lytic initiation, HSV1, CMV and EBV genomes localize to PML NB as sites for viral replication compartments prior to PML NB disruption (Bell et al., 2000, J. Virol. 74:11800-11810; Ishov and Maul, 1996, J. Cell Biol., 134:815-815-826; Ishov et al., 1997, J. Cell Biol., 138:5-16; Maul et al., 1996, Virology 217:67-75)

It would be useful to have ways to activate EBV in a manner that would permit reducing the population of EBV-infected cells in persons having a cancer or other condition related to an EBV infection. It would also be useful to have ways to activate other herpes viruses and reduce the population of herpes-infected cells in persons in need thereof.

### PARTIES TO JOINT RESEARCH AGREEMENT

Not applicable.

### REFERENCE TO SEQUENCE LISTING OR TABLE SUBMITTED ON COMPACT DISC AND INCORPORATION-BY-REFERENCE OF THE MATERIAL [SPECIFY NUMBER OF DISCS AND FILES ON EACH]

Not applicable.

### BRIEF SUMMARY OF THE INVENTION

In a first group of embodiments, the invention provides methods of reducing the number of Epstein-Barr virus (EBV)-positive cells in a subject diagnosed with an EBV-related condition, said method comprising co-administering effective amounts of an arsenic compound and of an anti-herpes virus antiviral agent, thereby reducing the number of EBV-positive cells in the subject. In some embodiments, the anti-herpes virus antiviral agent is administered before administration of the arsenic compound. In some embodiments, the anti-herpes virus antiviral agent is administered two hours or less before administration of the arsenic compound. In some embodiments, the anti-herpes virus antiviral agent is administered one hour or less before administration of the arsenic compound. In some embodiments, the anti-herpes virus antiviral agent is administered substantially simultaneously with administration of the arsenic compound. In some embodiments, the anti-herpes virus antiviral agent is administered within four hours after administration of the arsenic compound. In some embodiments, the anti-herpes virus antiviral agent is administered approximately two hours after administration of the arsenic compound. In some embodiments, the anti-herpes virus antiviral agent is administered approximately one hour after administration of the said arsenic compound. In some embodiments, the anti-herpes virus antiviral agent is administered immediately following administration of the arsenic compound. In some embodiments, the arsenic compound is selected from the group consisting of an inorganic arsenic compound and an organic arsenic compound. In some embodiments, the inorganic arsenic compound is arsenic trioxide. In some embodiments, the arsenic compound is administered intravenously. In some embodiments, the arsenic trioxide is administered intravenously. In some embodiments, the arsenic trioxide is administered at a dose of 9 to 45 μg/kg daily. In some embodiments, the arsenic trioxide is administered at a dose of 15 μg/kg daily. In some embodiments, the arsenic trioxide is administered at a dose of 0.75 to 5 μg/kg daily. In some embodiments, the arsenic trioxide is administered at a dose of 1.5 μg/kg daily. In some embodiments, the anti-herpes virus antiviral agent is selected from the group consisting of ganciclovir, cidofovir, acyclovir, famciclovir, and valaciclovir. In some embodiments, the anti-herpes virus antiviral agent is ganciclovir. In some embodiments, the ganciclovir is administered intravenously. In some embodiments, the EBV-related condition is selected from the group consisting of a cancer and EBV-positive idiopathic pulmonary fibrosis. In some embodiments, the cancer is selected from the group consisting of EBV-positive Hodgkin's lymphoma, Burkitt's lymphoma, nasopharyngeal carcinoma, post-transplant lymphoproliferative disease, AIDS-associated lymphomas, EBV-positive gastric cancer, EBV-positive breast cancer, and EBV-positive lymphoma. In some embodiments, the subject diagnosed with an EBV-related condition has not been previously been treated for, or is concurrently being treated for, acute promyelocytic leukemia with an arsenic compound. In some embodiments, if the subject diagnosed with an EBV-related condition has previously or concurrently been diagnosed with a malignant glioma that has been removed by surgery, that subject has not been treated with temozolomide. In some embodiments, if the subject diagnosed with an EBV-related condition has previously or concurrently been diagnosed with a metastatic endometrial cancer, the subject has not been treated with arsenic. In some embodiments, the subject diagnosed with an EBV-related condition has not previously or concurrently been diagnosed with small cell lung cancer. In some embodiments, the subject diagnosed with an EBV-related condition has not previously or concurrently been diagnosed with metastatic melanoma.

In another group of embodiments, the invention provides methods of reducing the number of cells infected with a herpes virus in a subject in need thereof, said method comprising co-administering effective amounts of an arsenic compound and of an anti-herpes virus antiviral agent, wherein said arsenic compound thereby reactivates latent herpes virus in the infected cells, and the anti-herpes antiviral agent interferes with replication of herpes virus in the infected cells in which the herpes virus has reactivated, thereby reducing the number of cells infected with said herpes virus in the subject. In some embodiments, the anti-herpes virus antiviral agent is administered two hours or less before administration of the arsenic compound. In some embodiments, the anti-herpes virus antiviral agent is administered one hour or less before administration of the arsenic compound. In some embodiments, the anti-herpes virus antiviral agent is administered substantially simultaneously with administration of the arsenic compound. In some embodiments, the anti-herpes virus antiviral agent is administered immediately after administration of the arsenic compound. In some embodiments, the anti-herpes virus antiviral agent is administered within one hour after administration of the arsenic compound. In some embodiments, the arsenic compound is selected from the group consisting of an inorganic arsenic compound and an organic arsenic compound. In some embodiments, the inorganic arsenic compound is arsenic trioxide. In some embodiments, the arsenic compound is administered intravenously. In some embodiments, the arsenic trioxide is administered at a dose of 9 to 45 μg/kg daily. In some embodiments, the arsenic trioxide is administered at a dose of 15 μg/kg daily. In some embodiments, the arsenic trioxide is administered at a dose of 0.75 to 5 μg/kg daily. In some embodiments, the arsenic trioxide is administered at a dose of 0.75 to 4 μg/kg daily. In some embodiments, the arsenic trioxide is administered at a dose of 0.75 to 3 μg/kg daily. In some embodiments, the arsenic trioxide is administered at a dose of 1.0 to 2 μg/kg daily. In some embodiments, the arsenic trioxide is administered at a dose of 1.5 μg/kg daily. In some embodiments, the subject in need thereof has not previously been treated with chemotherapy or radiation for a cancer. In some embodiments, the subject in need thereof has not been previously or concurrently been treated for with acute promyelocytic leukemia.

### BRIEF DESCRIPTION OF THE DRAWINGS

FIG. 1. FIG. 1 is a graph showing that Epstein-Barr Virus (EBV) infection in A549 cells (A549 is an adenocarcinomic human alveolar basal epithelial cell line) increases the intensity of immunofluorescence of PML NBs, while arsenic trioxide (ATO) disrupts this phenomenon. A549<EBV>3A (infected cells) or uninfected parental cells were treated with the indicated doses of ATO for 24 hr. The cells were then differentially stained to show the promyelocytic leukemia protein (PML) while the nuclei were stained with DAPI. PML nuclear bodies (NB) immunofluorescence intensity in lower magnification images (400×) was measured using ImageJ software. To account for variable cell number per field, the PML signal was normalized to DAPI signal and this PML/DAPI ratio was then normalized to uninfected CNE-1signal. Legend: ***=p<0.005 compared to uninfected control; ###=p<0.005 compared to A549<EBV-BX1> control without ATO. A549=uninfected parental cells; A549<BX1> are EBV-infected cells, “BX1” designates an antibiotic selection marker; ATO=arsenic trioxide.

FIGS. 2A and B. FIG. 2A shows a graph demonstrating that ATO increases the number of Zta positive cells in a dose dependent manner. Cells of two A549 cell lines infected with EBV, A549<EBV-BX1>3A and A549<EBV-BX1>2C, were treated with ATO for 3 days and imaged for Zta with one dye, while nuclei were stained with DAPI. FIG. 2A shows the count of ZTA positive cells from a minimum of 3 random slide fields expressed as a percentage of total cells. FIG. 2B. FIG. 2B shows that ATO induces EBV lytic cycle protein expression in a dose dependent manner. A549<EBV-BX1>3A cells were treated with the indicated doses of ATO for 24 h. Expression of EBV Zta and BMRF1 mRNA were quantified using qRT-PCR. BX1 is an antibiotic selection marker.

FIGS. 3A-C. FIGS. 3A-C show effects of ganciclovir (GCV) and arsenic trioxide (ATO) in EBV infected A549 cells. As shown in FIGS. 3A and B, ATO induced EBV lytic mRNA and protein expression is time dependent. For both Figures, A549<EBV-BX1>3A cells were co-treated with 10 nM ATO and/or 40 μM GCV as indicated for the indicated times. FIG. 3A is a graph of the relative fold change in Zta mRNA levels analyzed using qRT-PCR. FIG. 3B is a graph of the relative fold change in BMRF1 mRNA levels analyzed using qRT-PCR. FIG. 3C. FIG. 3C is a graph showing the results of immunofluorescent detection of ZTA in ATO (10 nM) and GCV (40 μM) treated A549<EBVBX1>3A and non-infected parental cells. ZTA was stained in red while nuclei were stained with DAPI. The cell number per wide field on day 8 of treatment was determined by DAPI count of 6 random slide fields and normalized to untreated cells. Cntl=control.

FIGS. 4A and B. FIGS. 4 A and B are graphs showing apoptosis as determined by annexin V/propidium iodide (PI) staining. Control and EBV-infected A549 cells were treated with ATO (10 nM) or GCV (40 μM) as indicated and stained for annexin V and propidium iodide. Samples were analyzed by flow cytometry. FIG. 14A: control cells. FIG. 14B: EBV-infected cells. Legend: A549: control cells. A549<EBV-BX1>: A549 cells infected with EBV and an antibiotic selection marker, BX1. Cntl=control. GCV=ganciclovir. ATO=arsenic trioxide. G/A=ganciclovir +ATO. PI=propidium iodide positive/annexin V negative; annexin V=annexin V positive/propidium iodide negative; (−/−)=negative for both propidium iodide and annexin V; (+/+)=positive for both propidium iodide and annexin V.

FIGS. 5A-D. FIGS. 5A-D show the effects of GCV and ATO on viral gene expression in cells of the nasopharyngeal carcinoma cell line CNE1 infected with EBV (designated as “CNE1<EBV-BX1>” cells). FIG. 5A is a graph showing the ATO dose response of CNE1<EBV-BX1> cells treated with the indicated for 24 h. EBV Zta and BMRF1 mRNA levels were quantified using qRT-PCR. FIG. 5B is a graph showing the ATO induced expression of EBV Zta in CNE1 cells. EBV-infected and parental CNE1 cells were treated with 1 nM ATO and/or 40 μM ganciclovir (GCV) for 8 days and stained for the immediate early EBV gene Zta, while nuclei were stained with DAPI. The graph presents the analysis of cell count from 10 random slide fields analyzed using the ImageJ nucleus counter and normalized to the uninfected non-treated cell count. Legend: CNE1=uninfected cells; CNE1<EBVBX1>=infected cells. ATO=arsenic trioxide. FIGS. 5C and D are graphs showing the relative expression of mRNA of two EBV proteins, Zta (FIG. 5C) and BMRF1 (FIG. 5D). Both Figures: CNE<EBV-BX1> cells were co-treated with 1 nM ATO and/or 40 μM GCV as indicated for 4 days. EBV Zta and BMRF1 mRNA levels were analyzed using qRT-PCR. For FIGS. 5 B, C and D, the symbols **=p<0.01 compared to uninfected; ***=p<0.005 compared to uninfected; and ###=p<0.005 compared to no GCV).

FIG. 6. FIG. 6 shows the effects of arsenic trioxide (ATO) and ganciclovir (GCV) co-treatment in an in-vivo model of nasopharyngeal carcinoma. 5×10⁶ CNE1<EBV BX1> cells were injected subcutaneously into the right flank of male nude mice and assessed for tumor formation. After 7 days of growth, animals were randomly assigned to 4 groups and treated once daily with either carrier control, GCV (100 μg/g), ATO (100 ng/g) or GCV (100 μg/g) +ATO (100 ng/g). Tumor volume was assessed on 3-4 day intervals and animals were sacrificed and tumors harvested at 21 days of treatment. Data is representative of 2 separate experiments, N=7. Legend: * means p=0.05, ** means p<0.01, *** means p<0.001.

FIGS. 7A-C. To determine if reactivation co-treatment strategy (GCV+ATO) translated to tumors of lymphoid lineage, experiments were carried out utilizing the established Burkitt's lymphoma-derived cell line MUTU1 and EBV-negative MUTU1<DNE1> cells. FIGS. 7A and B. MUTU1<DNE1>” cells (shown as “MUTU DN” in FIG. 7A) and MUTU1 cells (labeled as “MUTU wt” cells in FIG. 7B) were cultured in media containing either carrier control, 10 nM ATO or 100 nM ATO. Viable cells were assessed by a manual count and trypan blue exclusion at 2 and 3 days. Cells were harvested for protein and mRNA samples at day 3. FIG. 7A. No effect was noted in the EBV-negative cell line MUTU1<DNE1>. FIG. 7B. ATO treatment showed a dose and time dependent suppression of cell viability in the EBV-positive MUTU cells. FIG. 7C. FIG. 7C is a photo of a Western blot analysis of expression of the immediate early viral gene Zta in EBV-negative MUTU cells and EBV+MUTU cells. The EBV-negative “Mutu ANE1” cells show no Zta expression when contacted with 0, 10 nM, or 100 nM of arsenic trioxide (ATO), while the EBV+“MuTu wt” cells show Zta expression, indicating that the EBV lytic cycle has been reactivated in these cells.

FIGS. 8A-D. B-cells recently isolated from a patient were infected with EBV, allowed to grow out for several generations (only the EBV-infected cells would be able to grow out), and frozen until use. FIGS. 8A and B. FIGS. 8A and B graph the relative percent of viability of EBV-infected B-cells as just described (LCL<EBV1-13> cells) thawed and grown in one of: media alone (“control”), media and 45 μM ganciclovir (GCV), media and 0.1 nM arsenic trioxide (ATO), or media and a combination of these concentrations of GCV and ATO. The bars show the relative viability of cells after 2 days of this treatment (“2 D,” FIG. 8A), and at three days (“3 D,” FIG. 8B). FIG. 8C. Epstein-Barr nuclear antigen (“EBNA”) driven by the C promoter (“Cp”) is transcribed only during the latent phase of the virus. FIG. 8C graphs the relative expression of EBNA Cp mRNA of LCL<EBV1-13> cells grown three days (“3 D”) in media alone (“control”), media and 45 μM ganciclovir (GCV), media and 10 nM arsenic trioxide (ATO), or media and a combination of these concentrations of GCV and ATO. The results show that transcription of EBNA Cp mRNA drops dramatically in the presence of ATO, indicating that the virus has reactivated from the latent to the lytic phase. FIG. 8D. FIG. 8D graphs the relative transcription of the viral immediate early protein Zta mRNA of LCL<EBV1-13> cells grown three days (“3 D”) in media alone (“control”), media and 45 μM ganciclovir (GCV), media and 10 nM arsenic trioxide (ATO), or media and a combination of these concentrations of GCV and ATO. The results show that the transcription of Zta mRNA is approximately fourfold higher in cells contacted with ATO, indicating that the virus has reactivated from the latent to the lytic phase. Legend: * means p=0.05, ** means p<0.01, *** means p<0.001.

### DETAILED DESCRIPTION

### Introduction

### A. Epstein-Barr Virus-Related Conditions

As noted in the Background, Epstein-Barr virus (“EBV”) is a ubiquitous gamma herpes virus that has been implicated in a number of cancers, as well as in the progression of idiopathic pulmonary fibrosis. As also noted in the Background, for more than a decade, investigators have tried to find a method of inducing EBV to reactivate to the lytic form in infected cells so that the virus would be susceptible to anti-viral agents. Since 2000 alone, at least nine such attempts were reported in the scientific literature. These attempts employed a variety of approaches, including: radiation and sodium butyrate (Westphal et al., 2000, supra), 5-azacytidine (Moore et al., 2001, supra), cis-platinum, 5-fluorouracil, and taxol (Feng et al., 2002, supra), methotrexate (Feng et al., 2004a, supra); gemcitabine and doxorubicin (Feng et al., 2004b, supra, which also reports 5-azacytidine, cis-platinum, and 5-fluorouracil did not induce lytic EBV infection in EBV-transformed B cells in vitro and in vivo), rituximab and dexamethasone (Daibata et al., 2005, supra), valproic acid (Feng and Kenney, 2006, supra), 5-aza-2′-deoxycytidine and trichostatin A (Jung et al., 2007, supra), and richostatin A, sodium butyrate, valproic acid, and suberoylanilide hydroxamic acid (Hui and Chiang, 2010, supra).

Unfortunately, to date, none of these attempts to induce EBV lytic reactivation have proved to be a solution to the problem of reactivating EBV in EBV-positive cancer cells or in EBV-positive idiopathic pulmonary fibrosis so that some or all of the population of EBV-infected cells can be made susceptible to anti-herpes virus agents. This is a particular concern for EBV-positive cancers, such as EBV-positive Hodgkin's lymphoma, Burkitt's lymphoma, nasopharyngeal carcinoma, post-transplant lymphoproliferative disease, AIDS-associated lymphomas, EBV-positive gastric cancer, EBV-positive breast cancer, and EBV-positive lymphoma, as the EBV-infected cells tend to drive the progression of the cancer and reducing the population of such cells can be expected to reduce aggressiveness and progression. It is also a consideration for EBV-positive idiopathic pulmonary fibrosis, as the presence of EBV-positive cells is a marker for a poorer prognosis.

Surprisingly, we have now discovered that arsenic induces lytic phase reactivation of EBV in EBV-infected cells. Using an exemplar arsenic compound, arsenic trioxide, or “ATO,” studies underlying the invention found that arsenic reduced the concentration of promyelocytic leukemia protein nuclear bodies (“PML NBs”). Without wishing to be bound by theory, it is believed that the presence of PML NBs helps maintain EBV in its latent phase and that arsenic's effect in reducing PML NB concentration is related to the reactivation of EBV in arsenic-contacted cells, rendering them susceptible to killing with anti-herpes virus agents such as ganciclovir.

This discovery was then tested to determine whether it worked in an animal model. Surprisingly, as reported in the Examples, co-administration of an exemplar arsenic compound and an exemplar anti-herpes virus agent caused the size of human EBV+ nasopharyngeal carcinoma solid tumors in nude mice to shrink by almost half, while either agent by itself failed even to stop the tumors from growing larger. Thus, co-administration of an arsenic compound and an anti-herpes agent has surprisingly better effects on human EBV+ solid tumors than does either agent alone.

The finding that arsenic could reactivate latent EBV was then tested in both a long established EBV+ B cell line and in freshly obtained human B cells immortalized with EBV. As shown in the Examples, in both cases, arsenic was shown to induce the immediate early viral protein Zta, demonstrating reactivation of the virus. Further, as reported in the Examples, co-administration of both an exemplar arsenic compound and an exemplar anti-herpes virus agent was surprisingly better in reducing viability of human B cells freshly infected with EBV than was either agent administered alone. Thus, co-administration therapy is expected to have surprisingly better results in human EBV+ B cell conditions than either agent alone.

As EBV+ solid tumors and EBV+ B cell cancers and other conditions have different types of viral latency (for example, B cells have type 3 latency), we expect these results to be broadly applicable to EBV+ related conditions. Based on these results, we expect that co-administration of arsenic compounds and anti-herpes virus agents will result in reducing the population of EBV+ cells both in EBV+ solid tumors and in EBV+ related B cell conditions, such as post transplant lymphoproliferative disease and AIDS associated lymphomas. Accordingly, co-administration of these agents provides an important new strategy for stopping or slowing the progression of these disorders and, hopefully, for eliminating them or rendering them susceptible to elimination by more standard treatments.

Arsenic has a long and well known history of use as a poison. Arsenic trioxide was, however, approved by the Food and Drug Administration in 2000 as a treatment for relapsed or refractory acute promyelocytic leukemia (“APL”). For adults with relapsed or refractory APL, arsenic trioxide is typically administered intravenously over 1 to 2 hours at a dose of 0.15 mg/kg daily for 25 doses over a period up to 5 weeks. It is believed that administration of arsenic trioxide at this dosage induces apoptosis in APL cells.

Surprisingly, however, the effect in reactivating EBV occurs at arsenic levels an order of magnitude below that used for treating APL—the only currently FDA-approved use of an arsenic compound for therapy—and, even more surprisingly, the effect occurs at levels as much as two orders of magnitude below those levels. As the well known adverse cardiac and other side effects of arsenic are dose-related, embodiments of the invention exploiting this discovery therefore can render EBV-infected cells susceptible to treatment with anti-herpes virus agents at arsenic levels that substantially lower the possibility the subject will suffer adverse cardiac or other side effects related to standard doses. As arsenic trioxide use can prolong the QT interval, and potentially result in ventricular fibrillation, this is a substantial benefit.

Unlike the use of arsenic to treat APL, which is the only medical use for which it is currently approved by the FDA, in the methods of the invention, arsenic is not used to induce the death of the target cells. Rather, it is used to induce reactivation of a herpes virus, such as EBV, from the latent to the lytic phase, thereby rendering the virus susceptible to anti-herpes anti-viral agents. This difference in the mechanism of action allows arsenic to be administered in the methods of the invention at doses significantly lower than those needed for the treatment of APL. The studies underlying the invention show that arsenic can reactivate EBV at doses that are at least an order of magnitude lower than those used in the treatment of APL, and can be as much as two orders of magnitude lower. As arsenic compounds have dose-related side effects (including, at high doses, poisoning the subject), it is expected that lower doses of arsenic will reduce those side effects. Therefore, while doses of ATO currently used for treatment of APL (or equivalent doses of other arsenic compounds) can be used in the methods of the invention, in some preferred embodiments, the dosing of the arsenic compound is lowered by one order of magnitude, in some more preferred embodiments, between one and two orders of magnitude lower than the dose, and in even more preferred embodiments, is two orders of magnitude below those used in the treatment of APL. It should be noted that the lowered dose mentioned above refers to the amount of arsenic compound administered per kilogram of patient weight (e.g., for ATO, 0.15 mg/kg), not the cumulative amount of arsenic administered. In preferred embodiments, the arsenic compound is administered at 9 to 50 μg/kg daily, while in some embodiments, it is administered at 10 to 40 μg/kg daily, 11 to 35 μg/kg daily, 12 to 30 μg/kg daily, 12.5 to 25 μg/kg daily, 13 to 22.5 μg/kg daily, 13 to 20 μg/kg daily, 14 to 18 μg/kg daily, 14 to 17 μg/kg daily, or 15 μg/kg daily, with each successive dosage listed being more preferred. In other preferred embodiments, the arsenic compound is administered at 0.5 to 7.5 μg/kg daily, 0.75 to 7 μg/kg daily, 1 to 7 μg/kg daily, 1 to 6 μg/kg daily, 1.25 to 5 μg/kg daily, 1.25 to 4 μg/kg daily, 1.25 to 3 μg/kg daily, 1.25 to 2.5 μg/kg daily, 1.25 to 2 μg/kg daily, or 1.5 μg/kg daily, with each successive dosage listed being more preferred.

As noted above, in the inventive methods, the arsenic compound is administered not to kill the infected cells, but rather to induce reactivation of EBV from the latent to the lytic phase, thereby rendering the virus susceptible to an anti-herpes virus agent. The methods of the invention therefore involve co-administration of an anti-herpes virus agent so that a therapeutically effective level of the agent is present in the subject at the time the herpes virus is reactivated by the arsenic compound. Administration of an anti-herpes agent after the virus has reactivated is less effective at reducing the population of EBV-infected cells, as this allows for a period of lytic reactivation and production of infectious virions. The infectious virions can then infect more cells, thereby increasing the population of infected cells. Some of the newly infected cells will then enter the latent stage, rendering them unable to be killed by anti-herpes agents administered after the outbreak has caused clinical symptoms.

In studies underlying the present invention, we found that the virus reactivated in as few as 4 hours after being contacted with the arsenic compound, and can be as much as 24 hours afterwards. Accordingly, the anti-herpes agent should be administered so that a therapeutically effective amount is present in the blood no more than four hours after administration of the arsenic compound. Conveniently, this can be done by administration of the agent shortly before administration of the arsenic compound, about the time of administration of the arsenic compound, immediately after administration of the arsenic, or shortly thereafter, with the understanding that administration of the anti-herpes agent is preferably timed so that an effective blood level is present within four hours of the time administration of the arsenic was commenced. Administration of two therapeutic agents timed so that their intended effects overlap or are enhanced is well known in the art and variously termed “co-administration” or “combination therapy”. See, e.g., Shekhar Pandey et al., 2011, Arch Med Sci. 7 (5): 767-775; Connell and Saleh, 2012, Neurosci Lett. 507(1):43-46; Wakelee et al., 2012, Cancer Chemother Pharmacol. 69 (3):815-824; Boehm et al., 2010, Vet Anaesth Analg. 37 (6):550-556; Loibl et al., 2011, Breast Care (Basel) 6 (6):457-461, Powers, J., Clin. Infectious Dis. 39 (suppl 4):S228-S235.

Of the present anti-herpes virus agents, ganciclovir is among the most active against EBV and is accordingly preferred for use in connection with EBV-positive cancers or EBV-positive idiopathic pulmonary fibrosis. For use in the inventive methods, it is preferably administered intravenously. Conveniently, if the arsenic compound is administered intravenously, the ganciclovir may be administered through the same IV line as used for administration of the arsenic compound. Ganciclovir for intravenous use is commercially available (for example, under the trade name Cytovene®-IV from Genentech USA, Inc., S. San Francisco, Calif.), and directions for its use are well known in the art. For patients with normal renal function, it is typically administered at 5 mg/kg as a constant-rate infusion over 1 hour every 12 hours.

To provide an example of timing use intravenous arsenic trioxide as the exemplar arsenic compound and intravenous ganciclovir as the exemplar anti-herpes agent, if administration of the ATO is commenced at 9 a.m. (and typically continued over the next hour), administration of the ganciclovir could commence at 10 a.m. immediately after administration of the arsenic, or at any time thereafter until as late as 12:00 p.m., so that administration of the ganciclovir was concluded (and the ganciclovir therefore at full blood levels) by 1 p.m., four hours after the start of administration of the arsenic. This timing ensures that the anti-herpes agent is at an effective level by the time the herpes virus starts reactivating in response to the presence of the arsenic. The blood level of ganciclovir is then preferably maintained for 24 hours after administration of the arsenic or arsenic compound so that the agent is present for infected cells that have a reactivation lag-time of as much as 24 hours. As noted above, ganciclovir is typically administered every 12 hours. Accordingly, in the example set forth above, if the ganciclovir was administered commencing at 10 a.m., a second infusion of ganciclovir would be administered at 10 p.m. In some embodiments, the course of intravenous arsenic compound and intravenous anti-herpes agents is repeated once. In some embodiments, the course of intravenous arsenic compound and intravenous anti-herpes agents is repeated daily for a week. In some embodiments, the course of intravenous arsenic compound and intravenous anti-herpes agents is repeated daily for two weeks. In some embodiments, it is repeated daily or 5 times a week for a month. In some embodiments, particularly involving solid tumors, the arsenic and anti-herpes agent treatment may be continued for as long as 60 days to shrink the tumor and kill EBV-positive precursor cells.

In some embodiments, valganciclovir, a prodrug of ganciclovir with better oral bioavailability, is used in place of intravenous ganciclovir. The hydrochloride salt of valganciclovir (L-Valine, 2[(2-amino-1,6-dihydro-6-oxo-9H-purin-9-yl)methoxy]-3-hydroxypropyl ester, monohydrochloride) is commercially available under the name Valcyte® (Genentech, Inc.) both in 450 mg tablet form or in solution for oral use at a concentration of 50 mg/mL. For use in the inventive methods, it is contemplated that the dose would be 900 mg twice daily following administration of the arsenic compound. In some embodiments, the anti-herpes agent is cidofovir. In these embodiments, the cidofovir is administered at 5 mg/kg body weight (given as an intravenous infusion at a constant rate over 1 hr) at the same time in relation to the arsenic administration as described above. Since cidofovir is indicated for infusion only once a week, however, unlike ganciclovir, the infusion of cidofovir is not followed by a second infusion 12 hours later.

In some embodiments, the cancer is an early stage NPC with a homogenous population of EBV-infected cells. In some embodiments, the subject diagnosed with an EBV-related condition has not previously been treated for acute promyelocytic leukemia with an arsenic compound. In some embodiments, the subject diagnosed with an EBV-related condition has not previously been treated with an arsenic compound for a hematologic cancer or solid tumor. In some embodiments, if the subject diagnosed with an EBV-related condition has previously or concurrently been diagnosed with a malignant glioma that has been removed by surgery, said subject has not been treated with temozolomide. In some embodiments, if the subject diagnosed with an EBV-related condition has previously or concurrently been diagnosed with a metastatic endometrial cancer, said subject has not been treated with arsenic. In some embodiments, the subject diagnosed with an EBV-related condition has not previously or concurrently been diagnosed with metastatic melanoma. In some embodiments, the subject diagnosed with an EBV-related condition has not been treated with an arsenic compound to reduce the effects of chemotherapy or radiation. In some embodiments, the subject diagnosed with an EBV-related condition has not also been diagnosed as being immunocompromised. In some embodiments, the subject diagnosed with an EBV-related condition has not been exposed to high levels of arsenic by accidental arsenic poisoning, intake of Fowler's solution, intake of a supplement or therapeutic preparation containing an arsenic compound as an ingredient, exposure to high levels of arsenic by occupational exposure or in the environment, while taking an anti-herpes agent to prevent or to treat a herpes zoster occurrence or a herpes simplex outbreak. In some embodiments, the subject diagnosed with an EBV-related condition is not treated with the inventive methods with sodium arsenite, sodium meta-arsenite, or arsenic acid sodium salt for leukemia, non-small cell lung cancer, melanoma, renal cancer, uterine body cancer, colon cancer, gastric cancer, breast cancer, ovarian cancer, or prostate cancer, or has not taken an anti-herpes antiviral in the course of such treatment. In some embodiments, the subject has not taken realgar compound for chronic myeloid leukemia or has not done so while taking an anti-herpes antiviral. In some embodiments, the subject has not been treated before the administration of the arsenic compound with the nonsteroidal anti-inflammatory medication sulindac. In some embodiments, the subject is not taking an oral anti-herpes agent to treat herpes zoster or herpes simplex, or to suppress herpes simplex outbreaks, prior to administration of the arsenic compound. Arsenic is usually not detectable in the blood by normal tests. In some embodiments, if the subject has been exposed to high levels of arsenic, for example, by drinking for an extended period water having unusually high concentrations of arsenic, the subject is preferably not treated with the inventive methods until the subject's blood level of arsenic has returned to normal levels.

### B. Use For Herpes Viruses Other Than EBV

Arsenic has been used therapeutically for centuries. For example, Fowler's solution, a solution of arsenic trioxide in potassium bicarbonate, was used therapeutically for a variety of conditions from the 1700's into the early 1900's. See, e.g., Antman, 2001, The Oncologist 6 (suppl 2):1-2.) As stated in a 2004 letter to the editor by Lanska, Ann Hematol. 83:408, “an association between arsenic and herpetic lesions (both herpes zoster and herpes simplex) has been recognized since at least the 1880's with medicinal use of arsenic or with accidental or intentional arsenic poisoning.” [Citations within the quotation omitted]. In a notorious instance in 1900-01, 6,000 persons in and around Manchester, England, 70 of whom died, became ill with conditions variously diagnosed as alcoholic neuritis and beri-beri. It was finally discovered that the victims had been drinking beer accidentally contaminated with arsenic. See, e.g., Klatsky, 2006, Am. J. Epidemiol. 164 (2):194-195. A Dr. Reynolds, who tracked the epidemic to its source, reported that he and his staff had seen an extraordinary number of cases of alcoholic neuritis and noticed among these patients “a few cases of beautiful herpes zoster . . . Thereupon I remembered that arsenic was the only known drug which produced herpes, and so if there was any known drug acting as a poison in the beer it was almost certainly arsenic.” Reynolds, 1901, Med Chir Trans. 84: 409-452, at p. 411. Reynolds reported that out of 500 persons that he or his staff treated for arsenical poisoning, twenty one had herpes, id. at p. 429.

Herpes zoster (shingles) in particular was known as early as 1880 to be a side effect of taking medicinal arsenic and was thought to have been related to an immunosuppressant effect. See, Arsenic in Drinking Water, 1999, Subcommittee on Arsenic in Drinking Water, National Research Council, ISBN 978-0309063333 (National Academy Press, Washington, D.C.), at page 118. More recently, it was reported that 2 patients being treated with arsenic trioxide for APL developed herpes zoster some four weeks after treatment with ATO, but responded well to an oral antiviral. Tanvetyanon and Nand, 2004, Ann Hematol 83 (3):198-200. The occurrence of herpes zoster or herpes simplex in these and other reports in the literature are usually noted to have usually shown the weeks after administration of arsenic, or well after commencement of environmental or accidental exposure, and are often accompanied by hyperpigmentation and other effects of chronic low dose or acute high dose arsenic ingestion. Herpes labialis (cold sores caused by herpes simplex type 1) has been reported among the health effects of persons exposed to relatively high levels of arsenic in drinking water. For example, herpes labialis was reported by 32% of 114 adults in a 1982 Michigan health survey of persons exposed to arsenic in drinking water. Arsenic in Drinking Water, supra, at page 119.

As noted in the preceding section, studies underlying the present invention revealed that arsenic causes activation of a herpes virus of a different subfamily than that of varicella-zoster or herpes simplex, and that the activation occurs as early as four hours after exposure of infected cells to arsenic. Given the hundred and thirty year association between arsenic and occurrences of the alpha herpesviruses varicella-zoster and herpes simplex noted above, and the discovery underlying the present invention that arsenic reactivates immediate early proteins of the gamma herpesvirus EBV, it can be expected that arsenic causes reactivation of the alpha and beta herpes viruses, with a similar time course. Administering anti-herpes agents after an recurrence has become apparent to the subject, however, as in prior reports, may shorten the outbreak or reduce its symptoms, but will not serve to reduce the overall population of infected cells, and to the contrary will increase it, as the virus will have already replicated and infected additional cells by the time the outbreak is apparent to the sufferer. Accordingly, co-administration of arsenic and one or more anti-herpes agents on the time schedule described above with respect to EBV-related conditions is expected to reduce the population of cells infected by herpes viruses other than EBV in subjects in need thereof.

Shingles, or herpes zoster, outbreaks in persons infected with varicella-zoster virus can be quite painful and can substantially disable sufferers during an outbreak. Further, it can also result in postherpetic neuralgia, which can be experienced as a constant burning pain that may linger for weeks or months. Persons with a history of such outbreaks or at risk for one can particularly benefit from treatment according to the inventive methods. Similarly, persons with genital herpes, and particularly with genital herpes outbreaks that are not well controlled even with administration of acyclovir, valcyclovir or famcyclovir, will benefit from treatment according to the inventive methods.

As noted in the preceding section, in studies underlying the present invention, we found that the virus reactivated in as few as 4 hours after being contacted with the arsenic compound, and can be as much as 24 hours afterwards. Accordingly, the anti-herpes agent should be administered so that a therapeutically effective amount is present in the blood no more than four hours after administration of the arsenic compound. Conveniently, this can be done by administration of the agent immediately before administration of the arsenic compound, at about the time of administration of the arsenic compound, immediately after administration of the arsenic, or shortly thereafter, with the understanding that administration of the anti-herpes agent is preferably timed so that an effective blood level is present within four hours of the time administration of the arsenic was commenced. The arsenic compound can be administered in the dosages set forth in the preceding section.

Unlike EBV, varicella-zoster and herpes simplex types 1 and 2 are susceptible to treatment with acyclovir, valaciclovir, and famciclovir. In some preferred embodiments, the anti-herpes agent is acyclovir and both the arsenic compound and the acyclovir are administered intravenously, preferably with the acyclovir administered either immediately after administration of the arsenic compound. Acyclovir for intravenous infusion is commercially available in 20 mL and 40 mL vials, with each mL containing the equivalent of 25 mg of acyclovir. It is typically administered as a one hour infusion of 2.5 to 15 mg/kg and can be repeated every 8 hours. In preferred methods, therefore, intravenous acyclovir is administered no more than 3 hours after infusion of arsenic into the subject was commenced, and repeat acyclovir infusions are made at 8 and 16 hours thereafter to maintain effective blood levels of acyclovir. In some embodiments, the subject may be administered arsenic intravenously and famciclovir or valaciclovir orally. Valaciclovir and famciclovir have better oral bioavailability than does acyclovir, thus, higher blood levels can be achieved for these agents by oral administration than is possible with equivalent doses of acyclovir. If oral famciclovir is selected, it should be administered at 500 mg every 8 hours for 24 hours starting within two hours of commencing administering the arsenic compound to the subject. If valacyclovir is selected, 2 grams should be taken within two hours of commencing administering the arsenic compound to the subject and a further 2 grams 12 hours later.

In some embodiments, the subject diagnosed with a varicella-zoster or herpes simplex infection has not been treated for, and is not currently being treated for, acute promyelocytic leukemia with an arsenic compound. In some embodiments, the subject diagnosed with a varicella-zoster or herpes simplex infection has not previously been treated with, and is not concurrently being treated with an arsenic compound for a hematologic cancer or solid tumor. In some embodiments, if the subject diagnosed with a varicella-zoster or herpes simplex infection has previously or concurrently been diagnosed with a malignant glioma that has been removed by surgery, said subject has not been treated with temozolomide. In some embodiments, if the subject diagnosed with a varicella-zoster or herpes simplex infection has previously or concurrently been diagnosed with a metastatic endometrial cancer, said subject has not been treated with arsenic. In some embodiments, the subject diagnosed with a varicella-zoster or herpes simplex infection has not previously or concurrently been diagnosed with metastatic melanoma. In some embodiments, the subject diagnosed with a varicella-zoster or herpes simplex infection has not previously been treated with an arsenic compound to reduce the effects of chemotherapy or radiation. In some embodiments, the subject diagnosed with a varicella-zoster or herpes simplex infection has not also been diagnosed as being immunocompromised. In some embodiments, the subject diagnosed with a varicella-zoster or herpes simplex infection has not also been determined to having high blood or urine levels of arsenic due, for example, to exposure to high levels of environmental arsenic, to accidental arsenic poisoning, to intake of Fowler's solution, intake of a supplement or therapeutic preparation containing an arsenic compound as an ingredient, or by exposure to high levels of arsenic by occupational exposure. In some embodiments, the subject diagnosed with a varicella-zoster or herpes simplex infection has not been treated with the inventive methods with sodium arsenite, sodium meta-arsenite, or arsenic acid sodium salt for leukemia, non-small cell lung cancer, melanoma, renal cancer, uterine body cancer, colon cancer, gastric cancer, breast cancer, ovarian cancer, or prostate cancer or has not taken an anti-herpes antiviral during the course of such treatment. In some embodiments, the subject has not taken realgar compound for chronic myeloid leukemia or has not done so while taking an anti-herpes antiviral. In some embodiments, the subject has not been treated before the administration of the arsenic compound with the nonsteroidal anti-inflammatory medication sulindac. In some embodiments, the subject has not taken a course of Fowler's solution in combination with an anti-herpes antiviral agent to reduce or stop recurrences of genital herpes. In some embodiments, the subject is not taking an oral anti-herpes agent to treat varicella-zoster or herpes simplex, or to suppress varicella-zoster or herpes simplex outbreaks, prior to administration of the arsenic compound. Arsenic is usually not detectable in the blood by normal tests. In some embodiments, if the subject has been exposed to high levels of arsenic, for example, by drinking for an extended period water having unusually high concentrations of arsenic, the subject is preferably not treated with the inventive methods until the subject's blood level of arsenic has returned to undetectable levels.

### Definitions

Units, prefixes, and symbols are denoted in their Systeme International de Unites (SI) accepted form. Numeric ranges are inclusive of the numbers defining the range. Unless otherwise indicated, nucleic acids are written left to right in 5′ to 3′ orientation. The headings provided herein are not limitations of the various aspects or embodiments of the invention, which can be had by reference to the specification as a whole. Accordingly, the terms defined immediately below are more fully defined by reference to the specification in its entirety.

The phrase “therapeutically effective amount” refers to the amount of an active ingredient of a drug or other therapeutic agent which is sufficient to elicit the biological or medical result desired by the practitioner. Generally, the therapeutic result is an objective or subjective improvement of a disease or condition, achieved by inducing or enhancing a physiological process, blocking or inhibiting a physiological process, or in general terms performing a biological function that helps in or contributes to the elimination or abatement of the disease or condition.

“Co-administration” and “combination therapy” are used herein as synonyms and refer to the administrating to a subject two or more drugs or other therapeutic agents close enough in time so that therapeutically effective amounts of each of the drugs are present at the subject at the same time. The drugs or other therapeutic agents can be administered by the same route or by different routes. For example, a first therapeutic agent of the combination selected may be administered by intravenous injection while the other therapeutic agent of the combination may be administered orally. Alternatively, for example, all therapeutic agents may be administered orally or all therapeutic agents may be administered by intravenous injection.

“Substantially simultaneously,” with respect to administration of two or more drugs, refers to administering the drugs at the same time or within a few minutes (plus or minus five) of each other. For example, a subject might be administered one drug by intravenous infusion over an hour, while receiving a second drug orally at a point in time during the intravenous infusion, or might receive one drug orally just before a second drug is administered intravenously.

“Approximately,” with respect to a stated time for administering a drug, means within 15 minutes plus or minus of the stated time.

As used herein, “anti-herpes agent,” “anti-herpes agent,” and “anti-herpes antiviral agent” are all synonyms and refer to a drug that kills one or more herpes viruses or specifically interferes with their replication, and more specifically refers to agents that preferentially kill herpes viruses or inhibit their replication in vivo at levels that do not kill the majority of uninfected human cells.

“Arsenic” refers to the chemical element of that name. With respect to the therapeutic uses discussed herein, it will be understood that arsenic is not administered therapeutically as an isolated element but rather as part of a chemical compound, such as arsenic trioxide. Accordingly, unless otherwise required references herein to administration of “arsenic” refer to appropriate compounds of arsenic.

### Herpes Viruses

The family Herpesviridae comprises dozens of viruses, divided into several subfamilies. At present, only 8 herpes viruses, designated human herpes viruses 1 to 8, are known to infect humans. Of particular clinical relevance are:
human herpes virus 1, otherwise known as herpes simplex type 1, an alpha herpesvirus; human herpes virus 2, otherwise known as herpes simplex type 2, an alpha herpesvirus; human herpes virus 3, otherwise known as varicella-zoster virus, or “VSV” (the causative agent of shingles, which is sometimes referred to as “zoster” or as “herpes zoster”, as well as chicken pox), also an alpha herpesvirus;
human herpes virus 4, otherwise known as Epstein-Ban Virus, or “EBV,” a gamma herpesvirus;
human herpes virus 5, otherwise known as cytomegalovirus, or “CMV,” a beta herpesvirus; and
human herpes virus 8, otherwise known as Kaposi's sarcoma-associated herpes virus, or “KSHV,” a gamma herpesvirus.

As reported in U.S. Pat. No. 6,632,445, HSV virions are large, enveloped viruses with icosahedral capsids. They have double stranded linear DNA with a genome that encodes at least 70 polypeptides. This large amount of regulatory information permits the virus to control its own gene expression and to modify multiple complex events within the infected cell.

Importantly for the present invention, herpes viruses have a latent phase, in which they persist in a quiescent form, usually in neural ganglia. During this phase, the virus is not actively replicating and is not susceptible to anti-herpes virus agents which act by interfering with the viral polymerase. Latent herpes viruses can reactivate to the lytic stage or phase, in which the virus replicates. It is only during replication in which anti-herpes agents such as nucleoside analogs can be effective.

The invading virion binds to host cell receptors. A primary binding site is host cell surface heparan sulfate glycosaminoglycan, which binds with the V3 loop of the viral envelope glycoprotein (gp 120). Another primary binding site may be chondroitin sulfate. Mediated by viral glycoprotein gB and following nonspecific primary binding, more specific binding occurs to the gC4 and gD4 viral surface glycoproteins. The virion envelope fuses with the plasma membrane of the host cell. The capsid is uncoated, the virus invasively inserts surface glycoprotein gB through the host cell plasma membrane and enters the host nucleus where viral DNA is transcribed and processed into mature mRNA; at the same time, host cell mRNA synthesis is inhibited. Invading HSV also inhibits host cell DNA synthesis while viral DNA replicates within the host nucleus. The viral DNA combines with newly formed HSV capsid proteins translated in the cytoplasm, and assembles into progeny virion particles within the nuclear membrane. Concurrent expression of glycoproteins in the host plasma envelope stimulates neighboring cells to clump together. Following cell-to-cell contact by binding and fusion of their respective plasma envelopes, progeny particles invade clumped, neighboring host cells directly or by spread following lysis of previously invaded tissue cells or phagocytes and the process repeats itself.

Viral invasion elicits a phagocytic response coupled with typical phagocytic immune Activities—the release of soluble immune mediators (i.e., cytokines) and high respiratory burst responses by activated phagocytes. These immune responses are themselves detrimental to the host; not only because of local tissue necrosis from high environmental levels of free radical release, but also because of the development of mutant, potentially resistant viral strains secondary to toxic local levels of activated oxygen and hydroxyl species.

### Anti-Herpes Antiviral Agents

A key enzyme in the replication of herpesviruses is the virus-coded DNA polymerase. Most of the currently available anti-herpes drugs target the viral DNA polymerase. These drugs fall into two general categories. The first group consists of nucleoside analog inhibitors of herpesvirus DNA polymerases. This group includes acyclovir, penciclovir, and ganciclovir (9-[[2-hydroxy-1-(hydroxymethyl)-ethoxy]methyl]guanine), and their prodrugs, such as famciclovir, which is a prodrug of penciclovir, and valacyclovir, which is the hydrochloride salt of the L-valyl ester of acyclovir. Generally, these drugs must first be phosphorylated to the monophosphate forms by virus encoded kinases and further phosphorylated to triphosphate by cellular kinases before they are active inhibitors. The triphosphate forms of these nucleoside analogs inhibit polymerases by competing with the binding of natural triphosphates and their subsequent insertion into growing DNA strands. Since viral thymidine kinase phosphorylates the drugs faster than do cellular kinases, concentrations of the active drug rapidly become higher in virally-infected cells than they do in normal cells. The parent drugs are commercially available in both generic and branded forms, while famciclovir and valacyclovir are commercially available under the brand names Famvir® and Valtrex®, respectively. Other nucleoside analogs that have activity against herpesviruses are the adenine analog vidarabine (9-β-D-ribofuranosyladenine), the thymine analog brivudine (5-[(E)-2-bromoethenyl]-1-[(2R,45,5R)-4-hydroxy-5-(hydroxymethyl)oxolan-2-yl]-1,2,3,4-tetrahydropyrimidine-2,4-dione), the uridine analogs idoxuridine (1-[(2R,45,5R)-4-hydroxy-5-(hydroxymethyl)oxolan-2-yl]-5-iodo-1,2,3,4-tetrahydropyrimidine-2,4-dione), trifluridine (1-[4-hydroxy-5-(hydroxymethyl)oxolan-2-yl]-5- (trifluoromethyl) pyrimidine-2,4-dione) and edoxudine (5-ethyl-1-[4-hydroxy-5-(hydroxymethyl)oxolan-2-yl]pyrimidine-2,4-dione), and the cytosine analog cytarabine (4-amino-1-R2R,3S,4R,5R)-3,4-dihydroxy-5-(hydroxymethyl)oxolan-2-yl] pyrimidin-2-one). Some if not all of these drugs can be administered orally or, if necessary, intravenously.

Ganciclovir, preferred especially in embodiments of the invention involving EBV+ cancers or EBV+ idiopathic pulmonary fibrosis, will typically be administered intravenously. The recommended initial dosage for patients with normal renal function is 5 mg/kg, given intravenously at a constant rate over 1 hour, every 12 hours for 14 to 21 days.

A second group of agents are non-nucleoside inhibitors of herpesvirus DNA polymerases. Foscarnet (phosphonoformic acid, trisodium salt), for example, is a non-nucleoside pyrophosphate analog that selectively inhibits the pyrophosphate binding site on viral DNA polymerases at concentrations that do not affect cellular DNA polymerases. It is known to inhibit herpesviruses, including cytomegalovirus and herpes simplex types 1 and 2. For AIDS patients with CMV retinitis, for example, it has been administered at 60 mg/kg every 8 hours for 3 weeks, followed by maintenance treatment with 90 mg/kg/day until retinitis progression.

Most of the anti-herpes virus agents mentioned above are commercially available. Prescribing information for each for treatment of herpes infections, including dosing and methods of administration, is readily available for each. While dosage and administration information is discussed herein with respect to some exemplar anti-herpes virus agents and their use in the inventive methods, it is noted that persons of skill are well familiar with the use of these agents.

Since both foscarnet and an alternative agent, cidofovir (1-[(S)-3-hydroxy-2-(phosphonomethoxy)propyl] cytosine dehydrate, commercially available under the name Vistide®), an acyclic nucleoside phosphonate analog, exhibit significant nephrotoxicity and must be administered intravenously, they are currently reserved for use in patients with immunosuppression due to AIDS and particularly those with infections resistant to acyclovir. Cidofovir, however, has also been shown to have activity in reducing EBV proliferation (Abdulkarim et al., Oncogene 22L2260-2271 (2003). Its nephrotoxicity is typically addressed by prehydrating the patient with intravenous saline. It is typically administered at 5 mg/kg over one hour, once a week for two weeks. To reduce excretion of cidofovir in the urine, it should be administered with probenecid (4-(dipropylsulfamoyl)benzoic acid).

It is noted that vidarabine is more toxic than other nucleoside analogs, while trifluridine is used primarily in the eye, and cytarabine, currently used for treatment of acute myeloid leukemia and acute lymphocytic leukemia, causes bone marrow suppression. Accordingly, these agents are less preferred in the methods of the invention.

Most of the agents listed above are known to vary in their activity against the various herpes viruses. For example, acyclovir is stated to be active against herpesviruses in the following descending order of activity: herpes simplex type 1, herpes simplex type 2, varicella zoster, Epstein-Barr virus and, finally, cytomegalovirus. It is expected that persons of skill are aware of the degree to which the various agents are active against, for example, Epstein-Barr virus and will select and dose accordingly the anti-herpes virus agent selected.

The methods of the invention further contemplate the use of two or more anti-herpes agents. Such combinations are known in the art. For example, Moore et al., 2001, supra, report on the sensitivity of EBV-positive Burkitt's lymphoma cells to combinations of high doses of acyclovir and penciclovir (62.5 to 500 microM) and to lower doses of ganciclovir and bromovinyl deoxyuridine (10 to 100 microM). Bromovinyl deoxyuridine, or “brivudin,” is an oral thymidine analog for treatment of herpes zoster. It is typically administered once daily at 125 mg. See, Keam et al., 2004, Drugs 64 (18):2091-7.

It is expected that as additional anti-herpes anti-virals become available, they will be able to serve, alone or in tandem with current anti-herpes antiviral agents, as anti-herpes agents in the methods of the invention.

### Arsenic Compounds

A variety of inorganic and organic arsenic compounds have been used in over the years for therapeutic purposes. More recently, a number of organic arsenic compounds have been developed and stated to be useful for therapeutic use.

With respect to inorganic arsenic compounds, for example, compounds that may be used include, but are not limited to: arsenic (III) oxide (As₂O₃, also known as arsensic trioxide), arsenic (V) oxide (As₂O₅, also known as arsenic pentoxide), arsenic (III) selenide (As₂Se₃), arsenic (II) sulfide (As₂S₂), arsenic (III) sulfide (As₂S₃), arsenic (V) sulfide (As₂O₅), arsenic (III) telluride (As₂Te₃), sodium arsenate (Na₂HAsO₄), potassium arsenate (KH₂AsO₄), and sodium arsenyl tartrate (NaC₄H₄AsO₆). As ATO displaces zinc in the zinc finger motif of PML, trivalent arsenic should have the highest affinity in the methods of the invention and is preferred.

The exemplar therapeutic organic arsenic compound is arsphenamine, famously developed by Erlich in the early 1900's and marketed under the name “Salvarsan,” and its more soluble form, neoarsphenamine. More recently, various groups have synthesized a variety of organic arsenicals (OA) for potential therapeutic use, including derivatives synthesized by Chen et al., 1976, Carb. Res. 50:53-62; Rosenthal and Zingaro, 1980, Phosphorus and Sulfur 9:107-116; and Chen et al., 1980, J Chemical Soc, Perkin Trans. 1, 2287-2293.

The organic arsenical melarsoprol (C₁₂H₁₅AsN₆OS₂, also called “Mel B” or “Arsobal”) is still manufactured for human use. It is not commercially available for sale in the United States or Canada, but is available in the context of treatment INDs. Melarsoprol is used as a treatment for trypanosomiasis, or African sleeping sickness (see, e.g., Bisser, et al., 2007, J Infect Dis., 195 (3): 322-329, Hamon, et al., 1990, Pharmacol Biochem Behay., 36 (4):831-5), for which it is typically administered to adults at 2-3.6 mg/kg/day IV×3 days, after 1 week at 3.6 mg/kg/day IV×3 days, and again after 10-21 days at 3.6 mg/kg/day. It is usually administered by low intravenous injection as a 3.6% solution in propylene glycol. Melarsoprol has also been evaluated for its antileukemic properties (see, e.g., International Published Application WO 99/24029, EP1002537). It was stated to have activity at least equivalent to that of arsenic trioxide against both APL and non-APL cell lines (e.g., Konig et al., 1997, Blood 90:562-570). A limited clinical study of melarsoprol was performed in patients with advanced leukemia (Soignet et al., 1999, Cancer Chemother. Pharmacol. 44:471-421). Treatment of 8 patients with a dosing schedule that had previously been used for treatment of central nervous system trypanosomiasis showed a response only in a patient with chronic lymphocytic leukemia. Side effects experienced by study participants suggested that the dosing used was too high for treating persons with advanced leukemia.

Other organic arsenicals have been isolated or synthesized for therapeutic applications. For example, Lu, U.S. Published Patent Application 20040028750 discloses the use of arsenic sulfides as anti-cancer agents. Zingaro, U.S. Published Patent Application 20060189687, discloses carboxylic acid and dicarboxylic acid arsenicals, and in particular S-dimethylarsino-thiosuccinic acid (MER1), S-dimethylarsino-2-thiobenzoic acid (SAL-1), and S-(dimethylarsino) glutathione (SGLU1), which the application states were shown to exert significant anti-cancer activity against a panel of human leukemia cell lines. The Zingaro application states that MER1, SAL1, and SGLU1 showed similar efficacy to arsenic trioxide, and that MER1 and SGLU1 showed low toxicity against nonmalignant blood mononuclear cells from normal donors.

### Administration of Arsenic Compounds

As noted earlier, arsenic trioxide is currently used for treating APL and a limited range of other cancers. The doses of arsenic trioxide administered for that purpose in the studies reported herein revealed that reactivation of herpes viruses could be effected at concentrations below those used for APL. Without wishing to be bound by theory, it is believed that the amounts of arsenic employed in treating APL are needed to induce apoptosis, whereas the amount of arsenic needed to induce activation of early herpes virus proteins is much less. Accordingly, the amount of arsenic or compounds containing arsenic used in the inventive methods can be much less than those needed to have clinical effect in protocols treating APL or other cancers. In some embodiments an appropriate dosage of arsenic and/or one or more compounds of arsenic is in the range of 1-30 μg/kg, 2-20 μg/kg, or 5-10 μg/kg. In some embodiments, the dosage of arsenic is in amounts that are not generally sufficient to treat APL.

### EXAMPLES

### Example 1

This Example sets forth materials and methods used in studies reported below.

### Cell Culture And Transfection

Human lung adenocarcinoma cells (A549; display properties of type II pneumocytes) were obtained from ATCC. CNE1 cells were obtained from Samuel H. Speck, (Emory University, Atlanta, Ga., USA) and have been previously described (LTVCI, 1978, Sci. Sin. 21:127-134; Sizhong et al., 1983, Int. J Cancer, 31:587-590). Retroviral transduction and generation of EBV positive epithelial cells have been previously described (Sides et al., Am J Respir Cell Mol Biol. 2010). Unless otherwise noted, the EBV positive A549 cells used were the line designated A549<EBVBX1>3A (BX1 designates an antibiotic selection marker). All cells were maintained in Dulbecco's minimal essential medium (Invitrogen, Eugene Oreg.) supplemented with 10% fetal bovine serum (Gemini Bio-Products, West Sacramento Calif.) and 100 units/ml penicillin and 100 μg/ml streptomycin (Invitrogen). For transient transfection, cells were plated in 8-chamber LabTek slides (Fischer Scientific, Pittsburgh, Pa.) at an initial density of 3×10⁴ cells per well and transfected with Lipofectamine® with Plus reagent (Invitrogen) according to the manufacturer's instructions. Lipofectamine® RNAiMax (Invitrogen) was used in reverse transfection in siRNA experiments according to manufacturer's protocol. Akata BX1 cells were generously provided by Lindsay Hutt-Fletcher, (LSU School of Medicine, Shreveport, La.) and were maintained in RPMI (Invitrogen) supplemented with 10% fetal bovine serum (Gemini Bio-Products), 10 units/ml penicillin and 10 μg/ml streptomycin (Invitrogen) and 500 μg/ml G418 (Invitrogen #10131).

### Plasmids And Reagents

Arsenic trioxide (ATO) (Sigma Aldrich, St Louis Mo., #311383) was used at a concentration of 10 nM for treatment of A549 derived cell lines and at 1 nM for treatment of CNE1 derived cell lines unless otherwise noted. Ganciclovir (GCV) (Sigma Aldrich, #G2536) was used at a concentration of 40 μM unless otherwise noted. The pcDNA*Flag*LMP1*wt vector was kindly provided by Nancy Rabb-Traub (UNC-Lineberger Comprehensive Cancer Institute, Chapel Hill, N.C.). The siRNA constructs utilized have been previously described (Oh et al., 2009, J Cell Sci 2613-2622) and were purchased from Qiagen (Valencia, Calif.).

### Western Blot

Nuclear and membrane cell lysate fractions were separated using the Qproteome Cell Compartment Kit (Qiagen, #37502) according to the manufacturer's directions. Briefly, 5×10⁶ cells were harvested in extraction buffer, then separated by centrifugation and the cytosolic fraction (supernatant) was removed. The remaining pellet was resuspended in extraction buffer, separated by centrifugation and the membrane fraction (supernatant) was removed. After nuclease treatment, the pellet was resuspended in 500 μl of extraction buffer, separated by centrifugation and the nuclear fraction (supernatant) was removed. Nuclear (PML and membrane (LMP1) fractions were combined with 4× Laemmli Buffer (240 mM Tris, 8% SDS, 40% glycerol, 10% 2-mercaptoethanol, 0.02% bromophenol blue) and 30 μg of protein per well were loaded in a 10 well NuPage® Mops 4-12% gradient minigel (Invitrogen). Proteins were separated by electrophoresis at 130 V for 1.5 hours then transferred for 1.5 hours at 30V to PDVF membrane (Invitrogen). Protein transfer was verified by Ponceau-S staining. Membranes were blocked in 5% BSA in PBST for 1 hr prior to application of specified primary antibody overnight at 4° C. Rabbit polyclonal primary antibody to PML (Santa Cruz Biotechnologies, Santa Cruz, Calif., #sc-5621) was used at a dilution of 1:200. Rabbit polyclonal primary antibody to β-Actin (Cell Signaling, #4967) was used at a dilution of 1:1000. Mouse monoclonal primary antibody to LMP1 (B D Bioscience, Bedford, Mass., #559898) was used at a dilution of 1:1000. The fluorophore conjugated secondary antibodies IRDye® 680 goat anti-mouse IgG (LiCor Biosciences, Lincoln, Nebr., #926-32220) or IRDye® 800CW goat anti-rabbit IgG (LiCor, #926-32211) were used at a dilution of 1:15,000. Membranes were imaged using the Odyssey® Infrared Imaging System (LiCor).

### Immunofluorescence Microscopy

A549 cells were plated on 8-chambered Lab-Tek slides at 3×10⁴ cells per well; chemical treatment began 24 hours after plating. At specified time points, cells were rinsed twice with PBS and fixed in 4% paraformaldehyde (freshly diluted from 16%, Electron Microscopy Science, Hatfield, Pa.) for 10 min at room temperature. The cells were then rinsed twice with PBS and permeabilized with 0.5% Triton™ X100 (Sigma) in PBS for 15 min at room temperature. Primary antibodies were applied at specified concentrations overnight in a humidified chamber at 4° C. Following 3 rinses in PBS, secondary antibodies were diluted 1:500 and applied for 1 hour at room temperature. The chambers were removed and Vectashield® with DAPI (Vector Laboratories, Burlingame, Calif. #H1200) was applied with cover slip mount. The slides were imaged using a Nikon® Eclipse 80i microscope and a SensiCam QE camera (The Cooke Corporation, Auburn Hills, Mich.) and IPLab V3.65a software (Scanalytics, Inc., Rockville, Md.).

Exposure conditions were optimized for the brightest field of the specified conditions and held constant for subsequent exposures. The primary mouse monoclonal antibody (sc-966), and rabbit polyclonal antibody (sc5621) to PML (dilution 1:500) were purchased from SantaCruz. The primary monoclonal mouse antibody to LMP1 was purchased from BD Bioscience (#559898) and used at a dilution of 1:500. The primary antibody to ZTA (#11-007) was purchased from Argene Inc. (Shirley, N.Y.) and used at a dilution of 1:50. The secondary antibodies Alexa Fluor® 594 goat anti-mouse (Invitrogen #A11005), AlexaFluor® 594 goat anti-rabbit (Invitrogen#A21207) and AlexaFluor® 488 goat anti-mouse (Invitrogen #A11008) were used at a dilution of 1:500.

### Quantitative Reverse Transcription Real-Time PCR

For quantitative real time reverse transcriptase PCR (qRT-PCR), cells were plated in 6-well plates at 3×10⁵ cells per well. Cells were harvested at 24 hours post treatment unless otherwise noted. RNA was isolated using the Qiagen RNeasy Mini Isolation kit as per the manufacturer's specification. Briefly, cells were harvested in RLT buffer plus beta-mercaptoethanol. Samples were homogenized for 30 seconds at maximum rpms using a Tissuemiser homogenizer (Fisher Scientific). RNA concentration was quantified by UV spectroscopy and 1 μg RNA of each sample was used in a 20 μl reverse transcription reaction using iScriptTM reverse transcriptase and buffer (BioRad Laboratories, Hercules Calif.). Equal volumes of cDNA per sample were used in a 20 μl Syber® Green (BioRad) real-time PCR reaction in an iQTM thermal-cycle (Biorad). Target gene expression was normalized against 36B4 expression and control sample using the AACT formula and expressed as fold change. Primers were purchased from Integrated DNA Technologies (Coralville, Iowa). Primer sequences and run conditions for 36B4 (Shan et al., 2008, J Biol Chem, 283:21065-21073), ZTA and BMRF1 (Hilscher et al., 2005, Nucleic Acids Res, 33:e182) have been previously described.

### Cell Viability Analysis

Cell viability was assessed using the Annexin V-FITC apoptosis Detection kit purchased from Sigma (#APOAF) according to manufacturer's directions. Briefly, cells were plated at 3×10⁵ cells per well in 6-well culture dishes. At 24 hours, the culture medium was changed to DMEM+0.5% FBS and cells were treated as indicated. At 4 days post treatment, the cells were stained for annexin V and with propidium iodide and analyzed at the Tulane Center for Gene Therapy using the Beckman/Coulter FC500 flow cytometer and running CXP software.

### Statistical Analysis

Individual comparisons were analyzed by two-tailed unpaired t tests; multiple comparisons were analyzed by ANOVA with Modified Bonferroni post hoc test. A p-value <0.05 was considered significant. For figures, (*) denotes p<0.05 compared to control, (**) denotes p<0.01 compared to control, (***) denotes p<0.001 compared to control, (#) denotes p<0.05 ATO compared to ATO/GCV co-treatment, (###) denotes p<0.001 ATO compared to ATO/GCV co-treatment. Data presented is representative of multiple experiments performed in triplicate. Data is represented as the mean (+/−) SEM.

### Example 2

This Example sets forth results of studies undertaken in the course of the present invention.

### LMP1 Induced PML Protein Expression And Increased Nuclear Body Intensity

LMP1 expression in EBV-infected cells has been shown to inhibit lytic reactivation of EBV (Adler et al., 2002, Proc Natl Acad Sci U.S.A., 99:437-442). Previous studies have shown that PML expression is induced by interferon responsive elements and other studies have shown that LMP1 induces the expression of interferon responsive genes during EBV infection (Lavau et al., 1995, Oncogene, 11:871-876; Stadler et al., 1995, Oncogene, 11:2565-2573; Zhang et al., 2001, J Virol, 75:12393-12401). To test the hypothesis that LMP1 may regulate PML NB in a way that might support LMP1's anti-reactivation function, A549 cells were retrovirally transduced with either an empty backbone vector or the LMP1 expression vector, and were evaluated using immunofluorescent microscopy. The control cells displayed the expected punctuate PML NB staining. However, the difference in PML NB fluorescence intensity between the LMP1 positive and control cells was such that, when exposure time was optimized for the LMP1 positive cells, no signal was seen in the control cells. Quantification of PML NB intensity across the population of cells revealed a significant increase in PML NB fluorescence. To examine whether the increase in PML NB fluorescent intensity observed with LMP1 expression was due to an increase in PML protein expression rather than increased localization of PML protein to PML NB or PML NB aggregation, PML protein expression was examined by western blot. LMP1 expression resulted in an increase in all nuclear isoforms of the PML protein.

### LMP1 Modulation of PML NB Was Not Autocrine/Juxtacrine

To investigate whether the LMP1 induced increase in PML NB intensity occurs through an autocrine/juxtacrine mechanism, A549 cells were transiently transfected with the LMP1 expression plasmid or backbone vector and imaged with immunofluorescence microscopy 4 days later. LMP1 expressing cells displayed an increase in PML NB size and intensity when compared to either neighboring, non-transfected cells or cells transfected with the backbone plasmid, suggesting that PML upregulation is a direct effect of LMP1 expression within the cell. To corroborate these observations, A549 cells retrovirally transduced with LMP1 expression or empty vector were grown in trans-well co-culture with non-transduced, parental A549 cells. No upregulation of PML was observed in A549 cells grown in co-culture with LMP1 infected cells.

### LMP1 Expression In EBV Infected A549 Cells Resulted In Increased PML And PML NB Intensity

To investigate whether LMP1 expressed at physiological levels affects PML NB, A549 cells were infected with the BX-1 strain of EBV. Immunofluorescence imaging of EBV infected cells showed an increase in PML NB intensity when compared to uninfected parental cells, consistent with the results using LMP1 retrovirally transduced A549 cells.

### ATO Increased Expression of the Lytic Transactivator, ZTA, And Induced Expression of Lytic Proteins In EBV Infected A549 Cells

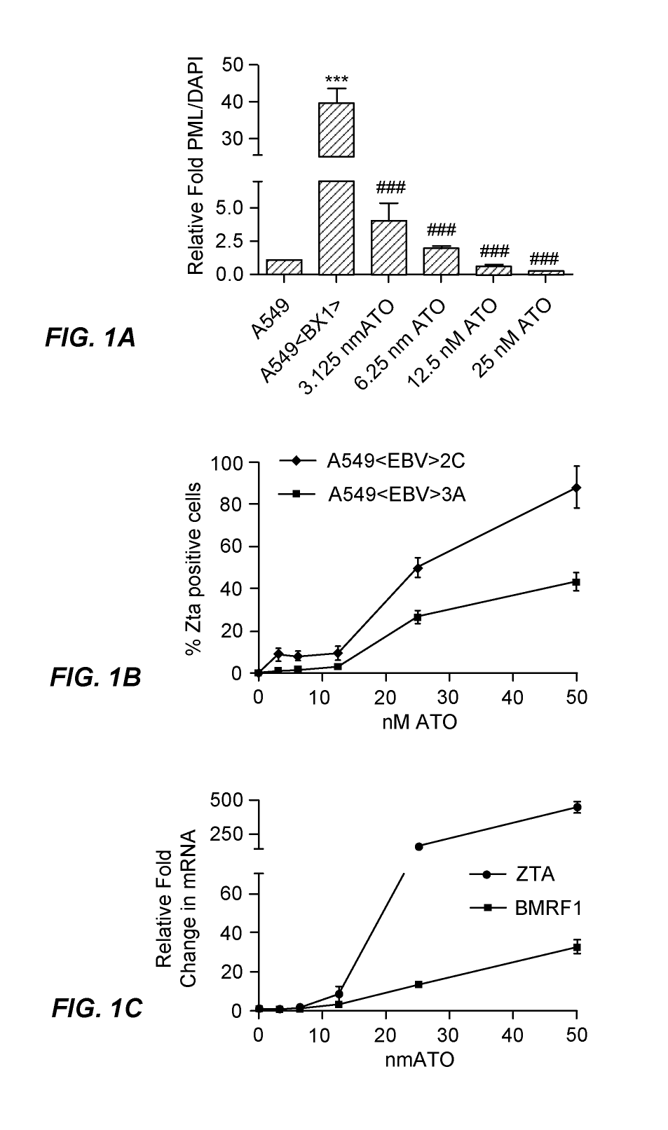

Expression of the EBV immediate early protein Zta has been shown to be sufficient to re-activate the lytic cycle in EBV infected cell, and has been shown to disrupt PML NB upon lytic reactivation (Adamson and Kenney, 2001; Speck et al., 1997). The increase in PML NB intensity by a latent viral protein and disruption of PML NB by IE lytic proteins suggest a possible role for PML NB in the maintenance of EBV latency. ATO has been shown to disrupt PML NBs by direct interaction with the consensus N-terminus RBCC domain of the PML protein (Zhang et al., 2010). To investigate the ability of ATO to disrupt PML NB in EBV infected A549 cells, cells were treated with varying doses of ATO for 24 hours and PML NBs were imaged. Treatment with ATO concentrations as low as 6.25 nM resulted in reduction of PML NB size, number and fluorescent intensity, and treatment with 12.5 nM ATO resulted in complete loss of PML NB immunofluorescence signal. Quantification of PML NB immunofluorescence intensity demonstrated that PML NB intensity was diminished by exposure to ATO in a dose dependent manner. Exposure to 12.5 nM ATO resulted in a PML NB immunofluorescence signal below that of baseline untreated uninfected cells. See, FIG. 1A. To assess whether EBV positivity affected PML protein levels, cell fractions from two separate EBV infections (A549<EBV>2C and A549<EBV>3A) and uninfected parental A549 cells were analyzed by western blot. LMP1 expression was confirmed in the membrane fraction of both EBV infected cell lines when compared to uninfected parental cells, and increased expression of PML was concordantly observed in the nuclear fraction. To investigate whether disruption of PML NB by ATO treatment was sufficient to induce expression of EBV lytic proteins, A549<EBVBX1>3A cells were exposed to varying doses of ATO for 72 hours and Zta was imaged using immunofluorescence. A physical count of the number of Zta positive cells from parallel experiments utilizing two separate EBV infections of A549 cells revealed an increase in the number of Zta positive cells in an ATO dose responsive manner in both cell lines. See, FIG. 1B. To quantify the increase in Zta expression, mRNA isolated from A549<EBVBX1>3A cells treated with varying doses of ATO for 72 hours was analyzed by qRT-PCR. ATO treatment induced an increase in Zta mRNA at ATO concentrations equal to or greater than 12.5 nM. See, FIG. 1C. Measurement of the transcript encoding the early protein BMRF1 indicated induction of the EBV lytic mRNA with doses equal to or greater than 12.5 nM within 72 hours.

### ATO Induced Expression of EBV Lytic Transcripts In A549 Cells And Conferred GCV Susceptibility

To further investigate the effects of ATO on Epstein-Ban early gene induction,

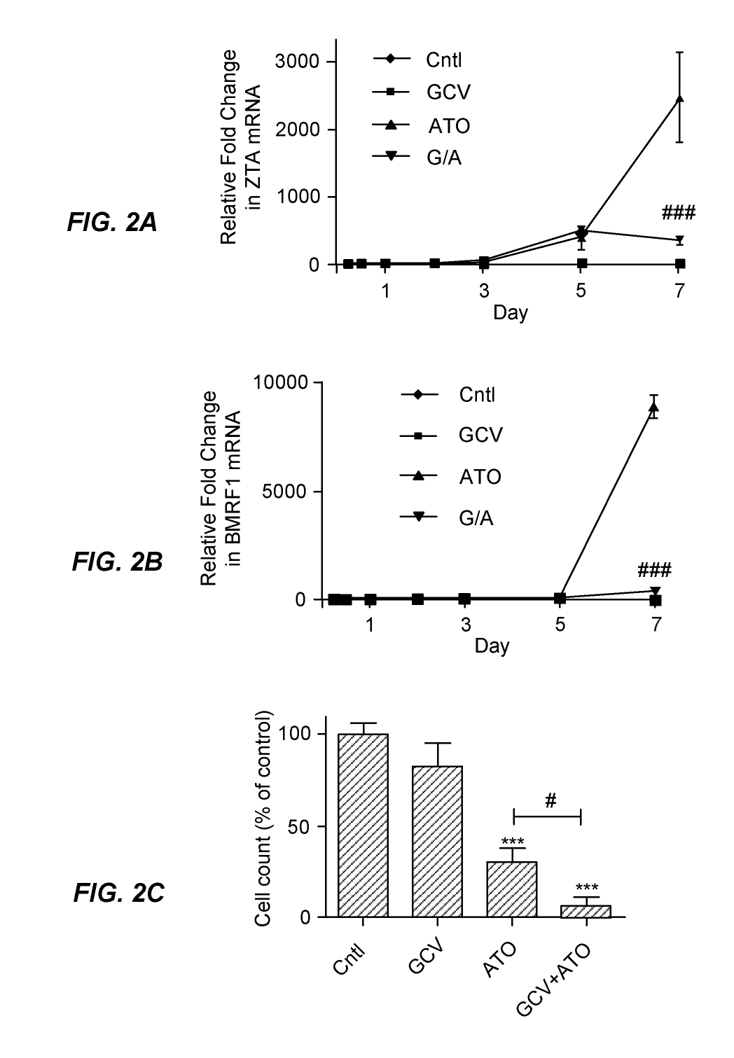

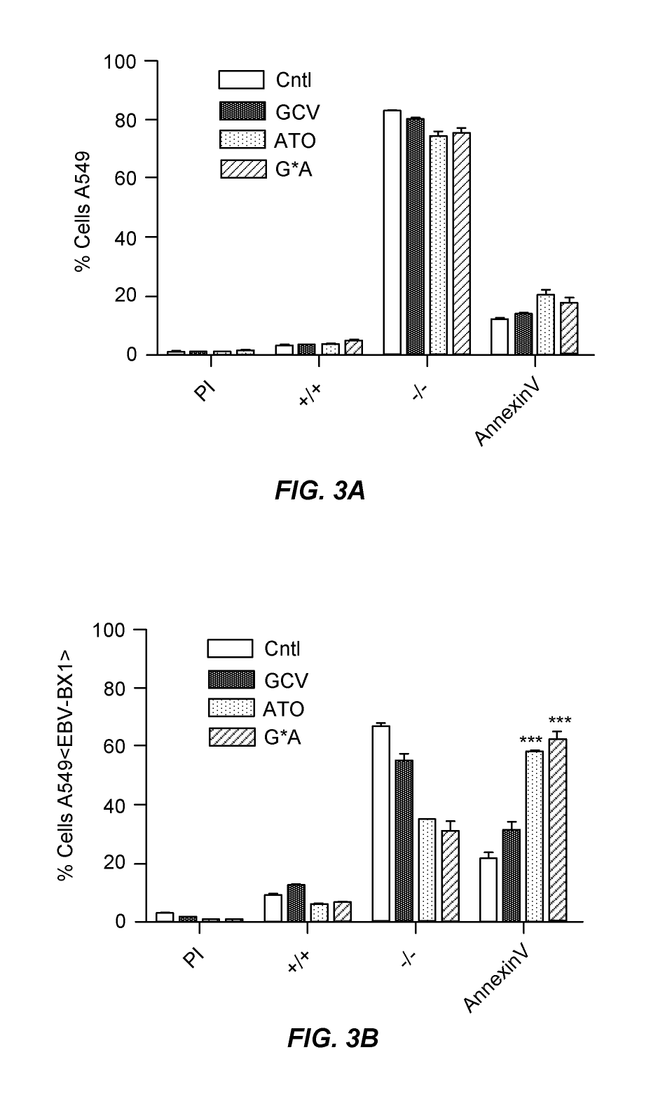

A549<EBVBX1>3A cells were treated with 10 nM ATO, in the presence or absence of GCV, and transcript levels of Zta and BMRF1 were assessed over 7 days using qRT-PCR. See, FIG. 2A. Treatment with 40 μM GCV alone had no effect on ZTA or BMRF1 mRNA expression. ATO treatment increased Zta mRNA expression as early as day 3, which increased even further over the ensuing 4 days. The observed increase in ZTA transcription was reduced at day 7 in GCV/ATO co-treatment samples when compared to ATO treatment alone. The increased levels of BMRF1 mRNA after ATO treatment lagged behind Zta expression, and this increase was abrogated by co-treatment with GCV/ATO. See, FIG. 2B. To quantify cell survival following ATO treatment and to assess susceptibility to GCV, A549<EBVBX1>3A and uninfected parental cells were treated with 10 nM ATO (plus or minus GCV) for up to 8 days and Zta was imaged using immunofluorescence at 2-day intervals. Zta expression was ATO responsive in a time dependent manner and 100% of the cells stained positively for Zta at day 8. Co-treatment with ATO and GCV also resulted in 100% of the cells staining positively for Zta as well as a reduction in the number of remaining cells. FIG. 2C. To assess whether the observed cell death required EBV infection, A549<EBVBX1>3A and uninfected parental cells were treated with GCV, ATO or GCV plus ATO for 4 days. The cells were stained for annexin V and with propidium iodide and viability was assessed using flow cytometry. FIGS. 3A and B. Treatment with GCV alone, ATO alone or co-treatment did not affect the viability of uninfected parental cells. In contrast, EBV positive cells underwent apoptosis when treated with ATO alone or with co-treatment. The percentage of viable cells was reduced from 67% to 35% and 31% with ATO and co-treatment respectively and there was a concurrent increase in the percentage of annexin V positive cells from 21% to 58% and 62%. While the difference between the ATO alone and ATO plus GCV is not statistically significant at this 4 day time point, there was a significant decrease in the number of surviving cells between these two groups at the 8 day time point.

### EBV Infection Increased the Intensity of PML NBs In NPC Cells

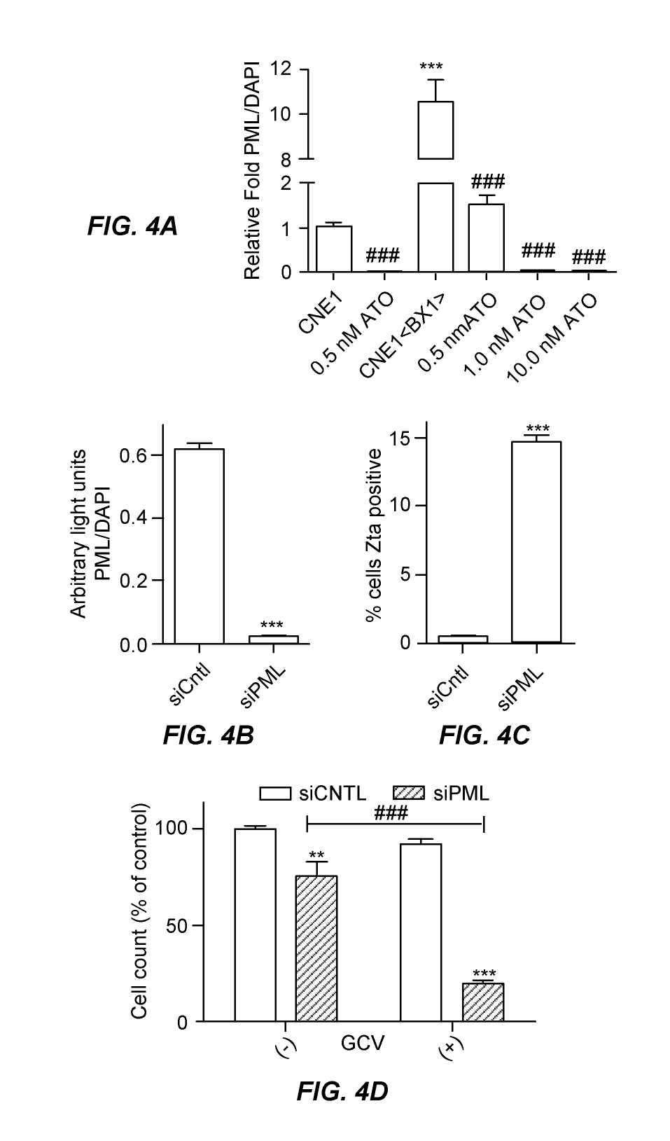

EBV infection is highly correlated with the development of anaplastic nasopharyngeal carcinoma (Nonoyama et al., 1973, Proc Natl Acad Sci U.S.A., 70:3265-3268; Raab-Traub, 2002, Semin Cancer Biol, 12:431-441; Tao and Chan, 2007, Expert Rev Mol. Med., 9:1-24; Thompson and Kurzrock, 2004, Clin Cancer Res, 10:803-821; Vasef et al., 1997, Ann. Otol. Rhino. Laryngol., 106:348-356; Wolf et al., 1973, Nat. New Biol., 244:245-247). To investigate the effect of EBV infection in NPC cells, CNE1 cells were infected with the BX-1 strain of EBV and PML-NBs were imaged by fluorescent microscopy. EBV infection increased the intensity of PML NBs when compared to non-infected parental cells, similar to the effect seen with EBV infection of A549 cells. Further, treatment with 1 nM ATO was sufficient to disrupt PML NBs in EBV positive CNE1 cells. Quantification of PML NB immunofluorescence intensity demonstrated that PML NB intensity was diminished by exposure to ATO in a dose dependent manner. Exposure to 1.0 nM ATO resulted in a PML NB immunofluorescence signal below that of baseline untreated uninfected cells. See, FIG. 4A.

### Disruption of PML NBs By siRNA Induces EBV IE Protein Expression In NPC Cells

To more specifically investigate the effects of disruption of PML NB on ZTA expression, siRNA directed against the PML transcript or a control siRNA were transfected into CNE1<BX1> cells. At day 4 post transfection, PML and Zta expression were analyzed using immunofluorescence. PML NB immunofluorescence intensity was greatly reduced in the majority of EBV positive CNE-1 cells transfected with PML siRNA. FIG. 4B. Consistent with the effects observed with disruption of PML NBs in response to ATO treatment, disruption of PML NBs using siRNA resulted in an increase in the number of cells expressing Zta. FIG. 4C. EBV infected and uninfected parental CNE1 cells were transfected with siRNA against the PML transcript or control siRNA cultured in the presence or absence of 40 μM GCV for 7 days and cells were counted in order to investigate whether disruption of PML NBs using siRNA induced GCV susceptibility. Treatment of PML specific siRNA transfected cells with GCV resulted in a reduction of cells to 20.1% (+/−1.5%) of non-treated EBV positive control cells. FIG. 4D.

### ATO Treatment Induced Expression of EBV Lytic Cycle Transcripts In NPC Cells

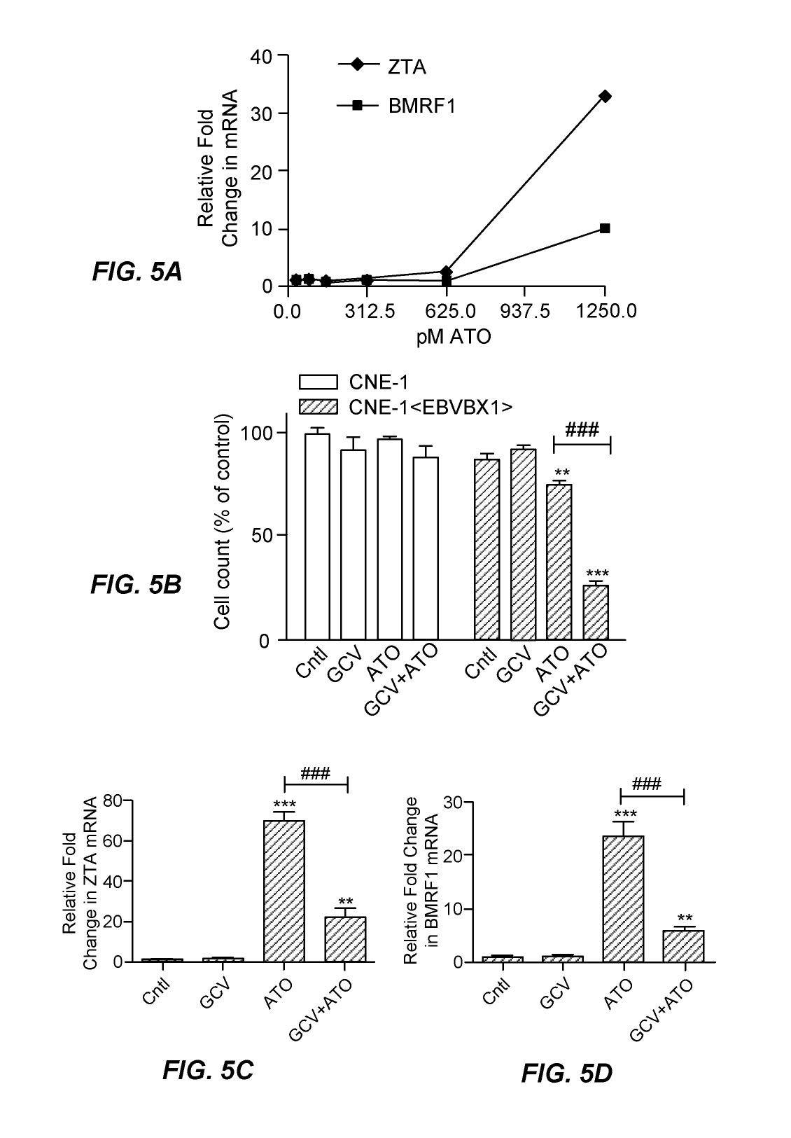

Current antiviral therapies directed toward EBV positive tumors require expression of EBV lytic proteins in order to confer susceptibility to antivirals (Kenney, 2006, Trans. Am. Clin. Climatol. Assoc., 117:55-73, discussion 73-74). To investigate the ability of ATO to induce expression of the EBV lytic transcripts in nasopharyngeal carcinoma cells, CNE1<EBV-BX1> and non-infected parental cells were treated with varying doses of ATO for 48 hours and Zta and BMRF1 mRNA levels were measured using qRT-PCR. See, FIG. 5A. Doses of ATO as low as 1.25 nM induced upregulation of ZTA, and to a lesser extent BMRF1, indicating induction of the early stage of EBV lytic transcription. CNE1<EBV-BX1> and non-infected parental cells were then treated with 1 nM ATO with or without 40 μM GCV for 3 days and ZTA positive cells were imaged using immunofluorescence. ATO alone and ATO plus GCV treatment increased the number of cells that were Zta positive.

### ATO Treatment In EBV Positive CNE1 Cells Conferred GCV Susceptibility And Induced Cell Death

EBV infected and uninfected parental CNE1 cells were treated with 1 nM ATO and 40 mM GCV or co-treated for 4 days and cells were counted to investigate whether ATO treatment induced GCV susceptibility. Co-treatment with GCV and ATO resulted in a reduction of cells to 26.0% (+/−5.4%) of non-treated EBV positive control cells. In a parallel experiment, EBV-positive CNE1 cells were treated with GCV, ATO, or GCV plus ATO for 4 days and ZTA and BMRF1 mRNA expression were quantified using qRT-PCR. Both Zta and BMRF1 mRNA expression were increased with ATO treatment when compared to control. With co-treatment, both Zta and BMRF1 mRNA levels were increased, but were well below that observed with ATO alone.

### Example 3

This Example discusses the results set forth in the previous Example.

The ability of EBV to evade the host immune system and antiviral therapy during the latent phase of the EBV lifecycle contributes to the ubiquitous nature of the virus. During latency, few viral proteins are expressed, major histocompatibility I expression is inhibited (Sengupta et al., 2006, Cancer Res., 66:7999-8006), and antigen processing and MHC class-I presentation of EBNA1 (Levitskaya et al., 1995, Nature, 375:685-688) is interrupted, suppressing the host adaptive immune response. Expression of LMP1 has been previously shown to suppress reactivation of the EBV lytic cycle as well as increase expression of interferon stimulated genes (Adler et al., 2002, Proc Natl Acad Sci USA, 99:437-442; Zhang et al., 2004, J Biol. Chem., 279:46335-46342). PML, as well as several PML NB components, are interferon stimulated genes that are thought to contribute to the innate host response to viral infection (Bernardi and Pandolfi, 2007, Nat. Rev. Mol. Cell Biol., 8:1006-1016). In this study, exogenous expression of LMP1 increased PML protein expression, along with the intensity of PML NB immunofluorescence. Additionally, upregulation of PML NBs was observed in EBV infected lung epithelial cells and nasopharyngeal carcinoma cells expressing LMP1. Measurement of the increase of PML NB intensity can be difficult since PML levels and PML NB number are known to fluctuate through the cell cycle and under injurious conditions (Ching et al., 2005, J Cell Sci., 118:847-854; Dellaire et al., 2006, J Cell Sci., 119:1026-1033; Eskiw et al., 2003, J Cell Sci., 116:4455-4466). The upregulation of PML in this study, however, was made apparent by the fact that the PML NB signal was more than an order of magnitude higher in LMP1 expressing cells.

Disruption of PML NBs plays a critical role in the life cycle of herpes viruses. HSV1-ICP0, CMV-IE1 and EBV-Zta disrupt PML NB at initiation of acute infection or lytic reactivation (Adamson and Kenney, 2001, J Virol., 75:2388-2399; Ahn et al., 1998, Mol. Cell Biol., 18:4899-4913; Bell et al., 2000, J Virol., 74:11800-11810; Bowling and Adamson, 2006, Virus Res., 117:244-253; Everett and Maul, 1994, EMBO J., 13:5062-5069; Kelly et al., 1995, J. Genl. Virol., 76 (Pt. 11)2887-2893; Maul and Everett, 1994, J Gen. Virol., 75 (Pt. 6):1223-1233; Xu et al., 2001, J Virol., 75:10683-10695). The conservation of this mechanism throughout the herpesviridae family suggests the importance of PML NB disruption at the initiation of the lytic replication cycle. The lytic reactivation of some herpes viruses with either environmental arsenic exposure, as well as, the use of ATO in the treatment of cancers has been previously reported (Ardalan et al., 2010, Clin. Cancer Res., 16:3019-3027; Au and Kwong, 2005, J. Am. Acad. Dermatol., 53:890-892; Bartolome et al., 1999, Br. J. Dermatol., 141:1106-1109; Boom et al., 1988, J Gen. Virol., 69(Pt. 6):1179-1193; Nouri et al., 2006, J. Drugs Dermatol., 5:182-185; Tanvetyanon and Nand, 2004, Anhn. Hematol., 83:198-200; Uede and Furukawa, 2003, Br. J. Dermatol., 149:757-762), although the reactivation of herpes viruses was not emphasized in the clinical trials. In order to investigate the role of PML NBs in viral latency in the context of a therapeutically viable method for treatment of EBV, PML NBs were targeted for degradation using ATO. ATO was previously shown to induce the degradation of PML by selectively binding to zinc finger motifs in the consensus RBCC domain and promoting polyubiquitination (Lallemand-Breitenbach et al., 2008, Nat. Cell Biol., 10:547-555; Weisshaar et al., 2008, FEBS Lett., 582:3174-3178; Zhang et al., 2010, Science, 328:240-243). ATO may, however, have off target effects involving other sumoylated proteins and pathways (Miller, 2002, Oncologist, 7 (Supp 1):14-19; Miller et al., 2002, Cancer Res., 62:3893-3903). Regardless, treatment of cells with ATO at nanomolar concentrations efficiently reduced the presence of PML NBs in A549 and CNE1 cells, and induced EBV lytic cycle protein expression in infected A549 and CNE1 cells. Additionally disruption of PML NBs by PML directed siRNA induced expression of the IE gene Zta and induced GCV susceptibility in EBV infected nasopharyngeal carcinoma cells. ATO was more uniform compared PML siRNA in inducing ZTA expression within the CNE1 cells, which may be attributable to the less than complete disruption of PML NBs using an siRNA knockdown strategy, compared with the more robust dissociation of PML NBs using ATO. Nevertheless, siRNA was as effective as ATO in promoting cell loss when used in combination with ganciclovir. We suspect that enough of the CNE1 cells underwent EBV reactivation with PML siRNA to induce sufficient ganciclovir activation to eliminate neighboring cells via a bystander effect.

Overexpression of EBNA1 has been shown to decrease PML at the protein level and PML NB number per cell through promoting phosphorylation and the polyubiquitination pathway (Sivachandran et al., 2010, J Virol., 84:11113-11123; Sivachandran et al., 2008, PLoS Pathog. 4:e10000170). This was shown convincingly in overexpression studies in EBV negative cells and in two experiments utilizing the EBV positive C666-1 cell line, which has been shown to be LMP1 negative (Cheung et al., 1999, Int. J. Cancer, 83:121-126). Though we did not specifically assess expression of EBNA1 in these cells, it is highly likely that EBNA 1 is co-expressed with LMP1. As previous studies have suggested that LMP1 is expressed in NPC (Edwards et al., 2004, J. Virol., 78:868-881; Hao et al., 2004, Otolaryngol. Head Neck Surg., 131:651-654), the model of newly infected CNE-1<EBV-BX1> cells utilized in this study is felt to be an accurate cell culture analogue of NPC. In this study, LMP1 expression alone or in the context of latent EBV infection increased the level of PML with a concomitant increase in PML NB immunofluorescence intensity. Disruption of PML NBs resulted in the expression of EBV lytic gene products. These data suggest a role for LMP1-mediated upregulation of PML and PML NBs in the maintenance of latency in EBV infection.

Current antiviral therapies targeting EBV positive tumors rely on phosphorylation of pro-drug nucleoside analogues by the viral lytic stage specific protein kinase (Meng et al., 2010, J. Virol., 84:4534-4542). Once phosphorylated, the nucleoside analogues inhibit viral genomic replication, and are also incorporated into the host genomic DNA inducing apoptosis (Freeman et al., 1993, Cancer Res., 53:5274-5283). Additionally, the activated pro-drug is transferred to neighboring cells and induces apoptosis through a “bystander effect” (Connors, 1995, Gene Ther. 2:702-709; Freeman et al., 1993, supra). This strategy is ineffective against the virus in the latent stage and requires exogenous reactivation of the viral lytic phase (Kenney, 2006, supra). Several agents have been utilized in conjunction with nucleoside analogues to confer susceptibility. The chemotherapeutics fluorouracil (5-FU), cis-platinum, gemcitabine, doxorubicin and methotrexate have been shown to induce EBV lytic cycle and confer GSV susceptibility in both lymphoid and epithelial EBV positive malignancies (Feng et al., 2004, J Natl. Cancer Inst., 96:1691-1702; Feng et al., 2004, J. Virol., 78:1893-1902; Feng et al., 2002, Cancer Res., 62:1920-1926). The broad histone deacetylase inhibitors sodium butyrate, valproic acid, trichostatin A (TSA), suberoylanilide hydroxamic acid (SAHA), the DNA methyltransferase inhibitor azacytidine, and gamma radiation have also been utilized to convey GCV susceptibility with varying, but suboptimal, results (Feng and Kenney, 2006, Cancer Res., 66:8762-8769; Hui and Chiang, 2010, J. Cancer, Int. J. Cancer, 126:2479-2489; Jung et al., 2007, Cancer Lett., 247:77-83; Moore et al., 2001, Antimicro. Agents Chemother., 45:2082-2091; Westphal et al., 2000, Cancer Res., 60:5781-5788). Rituximab, a chimeric antibody to CD20, in conjunction with the glucocorticoid, dexamethasone induced GCV susceptibility in lymphoma cells, though this strategy is specific to B-cell tumors (Daibata et al., 2005, J. Virol., 79:5875-5879). In addition, arginine butyrate selectively induces EBV TK with minimal lytic reactivation of EBV and has been used in clinical trials in conjunction with GCV in the treatment of post transplant lymphoproliferative disorder (Faller et al., 2001, Curr. Opin. Oncol., 13:360-367; Mentzer et al., 1998, Blood Cells Mol. Dis., 24:114-123).

In this study, ATO-induced expression of EBV lytic protein was shown to be time and dose responsive. Expression of EBV immediate early and early lytic proteins was achieved with nanomolar concentrations of ATO over an 8-day period. Perhaps the most critical aspect of our results from a potentially clinical standpoint is that after 8 days of exposure to ATO, Zta expression was observed in 100% of the cells. In EBV infected cells, ATO treatment induced apoptosis and conferred susceptibility to GCV treatment with a decrease in both cell number and BMRF1 expression in the remaining cells. Co-treatment with ATO and GCV resulted in a homogeneous Zta-positive population of cells, a statistical reduction in the number of cells, and reduced BMRF1 expression across the cell population when compared to ATO treatment alone. Taken together, these data indicate the efficacy of ATO and GCV co-treatment as therapy for EBV positive associated disorders. Importantly, at a concentration sufficient to induce expression of EBV lytic cycle protein expression profile in EBV positive cells, ATO had no effect on the viability of parental un-infected cells, indicating that ATO treatment can be a safe alternative to other methods of EBV reactivation that have been proposed for conferring susceptibility to antiviral treatment. In support of this concept, the dosage of ATO currently approved for treatment of relapsed or refractory acute promyelocytic leukemia resulted in peak serum arsenic level of ˜400 nM (Fox et al., 2008, Blood, 111:566-573). This level of ATO has also been proposed as a treatment option for relapsing or refractory multiple myeloma (Berenson and Yeh, 2006, Clin. Lymphoma Myeloma, 7:192-198; Rollig and Illmer, 2009, Cancer Treat. Rev., 35:425-430; Soignet et al., 2001, J. Clin. Oncol., 19:3852-3860). GCV is already approved by the FDA for antiviral treatment of CMV and EBV associated disorders. Our findings indicate that ATO can be a useful agent for inducing viral susceptibility in the host, and provides for the use of combination therapy with an arsenic compound, such as ATO, and an anti-herpes antiviral, such as GCV, on clearing viral loads and for treatment of EBV-associated malignancies.

### Example 4

This Example reports the results of in vivo results of human tumor xenografts in a murine model.

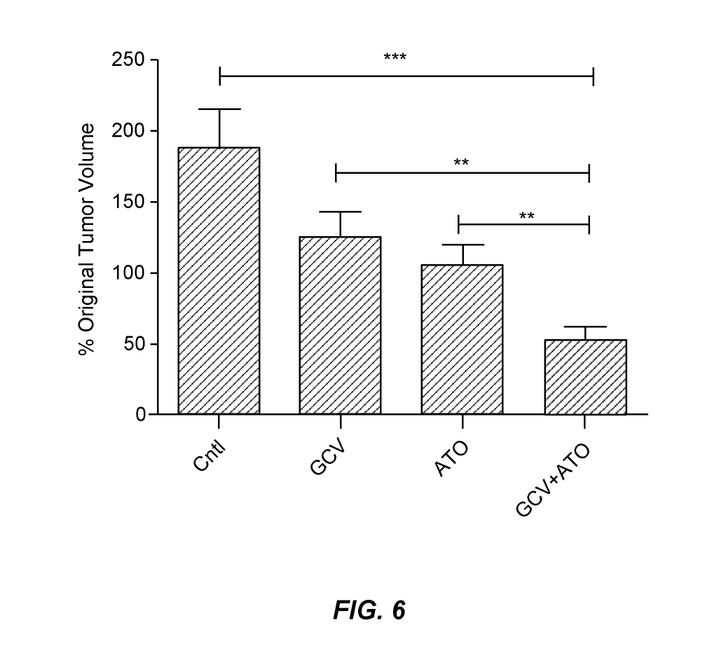

To investigate the effects of arsenic trioxide (ATO) and ganciclovir (GCV) co-treatment in an in vivo model of nasopharyngeal carcinoma, 5×10⁶ nasopharyngeal carcinoma cells infected with a strain of EBV carrying an antibiotic selection marker (CNE1<EBV BX1>cells) were injected subcutaneously into the right flank of male nude mice. The CNE1<EBV BX1> cells were maintained in media containing the selection marker until the day before injection into the mice to ensure a homogeneous population of EBV-infected cells. Seven days after the cells were injected into the animals, tumor volume was assessed, the animals were randomly assigned to 4 groups and then treated once daily with either carrier control, ganciclovir (GCV) at 100 μg/g, arsenic trioxide (ATO) at 100 ng/g, or with both GCV+ATO at these concentrations of. Tumor volume was assessed on 3-4 day intervals and animals were sacrificed and tumors harvested at 21 days of treatment. Data is representative of 2 separate experiments, N=7 for each treatment. FIG. 6 presents the data is graph form.

As can be seen in FIG. 6, after 21 days of the treatment noted (28 days after injection of the cells), the tumors in the control animals treated only with carrier had grown to a volume just under double their volume on day 7. In the animals treated with ganciclovir, after 21 days of treatment the tumors were approximately 25% larger than their size at day 7, showing that they had grown substantially, but more slowly than did tumors treated only with carrier. (Presumably the ganciclovir was inhibiting the growth of EBV-infected cells that spontaneously reactivated to lytic phase during the treatment and therefore became susceptible to ganciclovir.) In the animals treated with arsenic trioxide, the tumors were approximately 10% larger than their size on day 7, showing that they had not grown markedly over their pre-treatment size, but had also not regressed. In the animals treated with a combination of GCV and ATO, however, the tumor size was approximately half of the pre-treatment size, showing that the combination of agents had caused a substantial regression of the solid tumor. Since neither ganciclovir nor ATO by itself even stopped tumor growth, the fact that the co-administration of the two induced substantial regression is a surprising result.

### Example 5

Tumors from the animals in Example 4 were harvested for mRNA samples. Quantitative reverse transcription real-time PCR analysis of EBV genes showed an increase in transcription of both the EBV immediate early lytic gene BZLF1 (translated to the Zta protein) and the Zta dependent early lytic gene BMRF1 (translated to the EBV DNA polymerase processivity factor).

Fixed slides of tumor explants tissue were analyzed for Zta expression by immunofluorescence detection. Only the occasional Zta positive cell was observed in the control of GCV treated samples. In the ATO treated samples, large pockets of reactivation as noted by clustered expression of Zta protein were observed. These pockets of reactivation were also observed in the GCV+ATO co-treatment samples, though both the total number of cells and the number of Zta positive cells were diminished.

LMP1 has been reported to upregulate the activation of Stat3. The PML gene has been reported to be responsive to interferon activation of Stat 3 by binding to internal ribosome entry site (IRES) and GAS elements in the PML promoter region. CNE1 cells retrovirally transduced with either the empty vector or encoding LMP1 were analyzed for Stat3 localization by immunofluorescence detection. In LMP1 null cells, Stat3 was found to be diffuse throughout the cell. In LMP1 positive cells, however, the Stat3 signal was markedly nuclear.

A series of transfection/inhibitor studies were carried out to investigate the mechanism of upregulation of the PML-NB by LMP1. LMP1 has been shown to constitutively activate phosphorylation of ERK1/2 and NFκB. Inhibition of KFκB by JSH23 did not affect LMP1 upregulation of PML NBs. Either inhibition of ERK phosphorylation by U0126 or Stat3 dimerization by Stattic, however, prevented the LMP1 dependent upregulation of PML NBs.

### Example 6

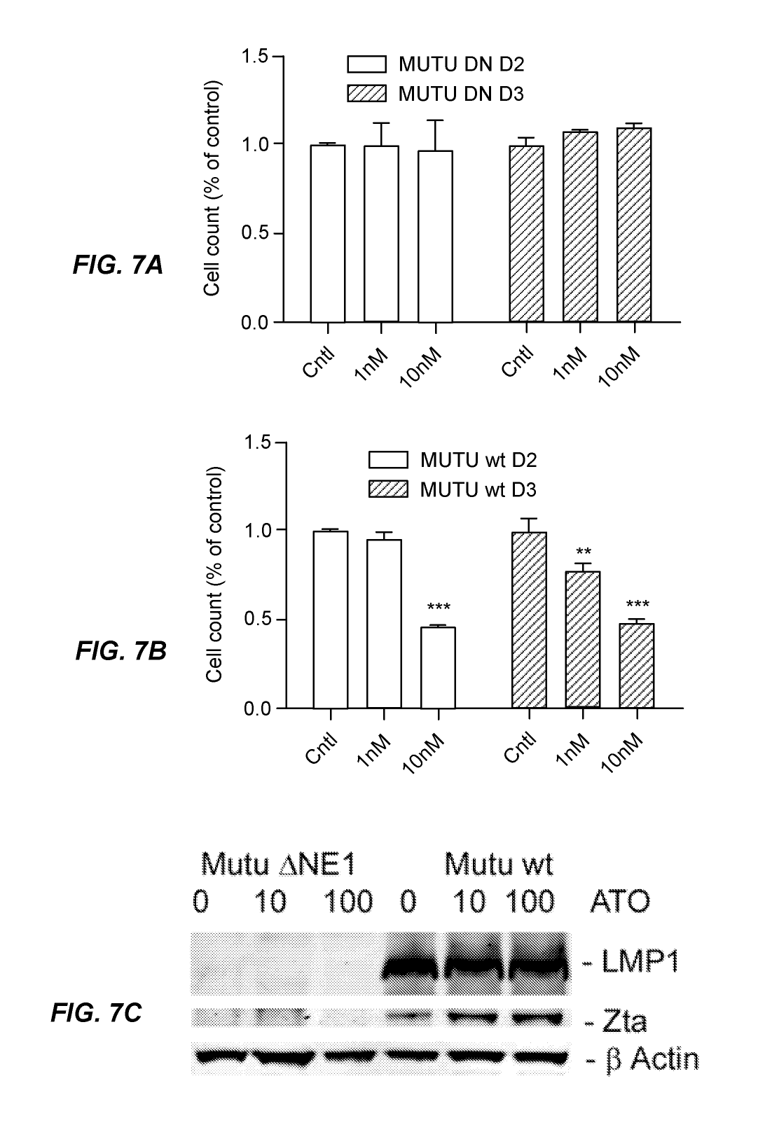

To determine if the reactivation co-treatment method strategy would apply to tumors of lymphoid lineage, a series of experiments was carried out utilizing an established Burkitt's lymphoma-derived cell line, MUTU1, and an EBV-negative variant, MUTU1<DNE1>. MUTU1 and MUTU<DNE1> cells were cultured in media containing either carrier control, 10 nM ATO, or 100 nM ATO. Viable cells were assessed by a manual count and trypan blue exclusion at 2 and 3 days. Cells were harvested for protein and mRNA samples at day 3. As shown in FIG. 7A, the two concentrations of ATO had no effect on the EBV-negative MUTU cells. In contrast, as shown in FIG. 7B, ATO treatment showed a dose and time dependent suppression of cell viability in the EBV positive cells. FIG. 7A legend: MUTU DN D2=MUTU EBV-negative cells, after 2 days of treatment indicated. MUTU DN D3=MUTU EBV-negative cells, after 2 days of treatment indicated. FIG. 7B legend: MUTU wt D2=MUTU EBV-positive cells after 2 days of treatment indicated; MUTU wt D3=MUTU EBV-positive cells after 3 days of treatment indicated. As shown in FIG. 7C, a Western blot analysis showed expression of the immediate early viral gene Zta was absent in MUTU EBV-negative cells (labeled “MUTU ANE1” in the Figure). In contrast, MUTU EBV-positive cells (labeled “Mutu wt” in the Figure) showed that ATO markedly enhanced reactivation of the EBV lytic cycle in MUTU1 cells.

### Example 7

To test the results obtained in Example 6 in a newly immortalized lymphoblastoid cell line (“LCL”), B-cells recently isolated from a patient were infected with EBV and grown out several generations to isolate cells that had been immortalized, resulting in a cell line designated “LCL<EBV1-13>.” The cells were cultured in media containing either carrier control (“Cntl”), 45 μM ganciclovir (GCV), 1.0 nM arsenic trioxide (ATO), or with both GCV and ATO at the just-stated concentrations. Viable cells were assessed by a manual count and trypan blue exclusion at 2 days (“2 D”) and 3 days (“3 D”). Cells were harvested for protein and mRNA samples at day 3.

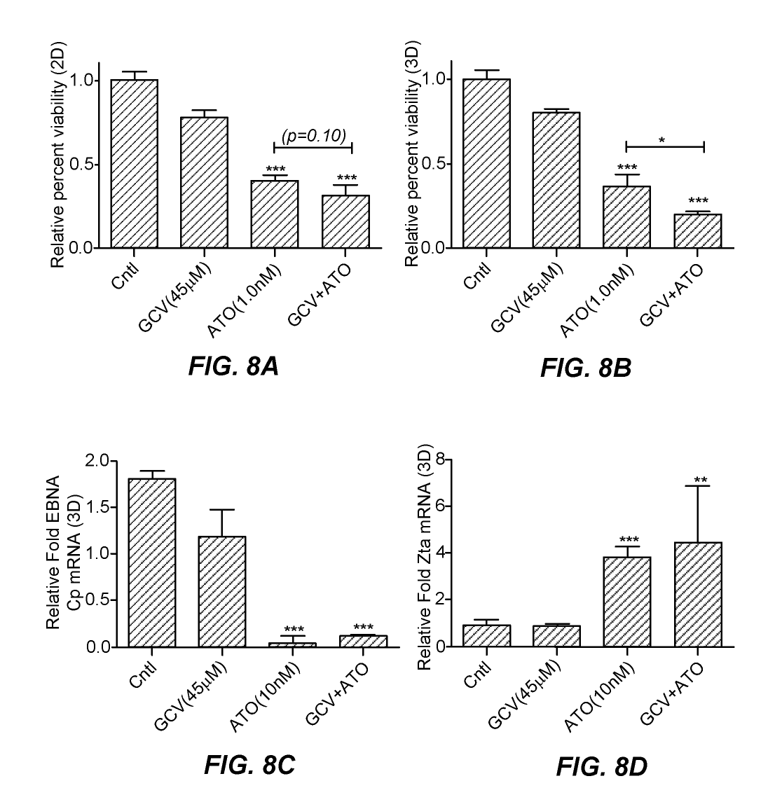

As shown in FIG. 8A, after two days of treatment, fewer cells remained viable after co-administration of GCV and ATO than after administration of ATO alone. FIG. 8B, however, shows that after a third day of treatment, the number of viable cells to which GCV+ATO has been co-administered dropped notably in comparison to those remaining viable after two days (compare FIG. 8B to FIG. 8A), whereas the number of viable cells treated with ATO alone barely changed. Further, the continued drop in viability over the additional day of co-administration could not be due to the effect of GCV by itself, as a comparison of FIGS. 8A and 8B shows that the further day of treatment with GCV did not result in reducing the number of viable cells compared to two days of treatment. The studies in the newly immortalized LCL cell line therefore confirmed that the effect of co-administration is greater than merely adding the effects of administering either agent alone. Moreover, on day four, there were no viable cells remaining in the co-administration group, while there were viable cells in the other treatment groups.

Epstein-Barr nuclear antigen (“EBNA”) driven by the C promoter (“Cp”) is transcribed only during the latent phase of the virus. FIG. 8C graphs the relative expression of EBNA Cp mRNA of LCL<EBV1-13> cells grown three days (“3 D”) in media alone (“control”), media and 45 μM ganciclovir (GCV), media and 10 nM arsenic trioxide (ATO), or media and a combination of these concentrations of GCV and ATO. The results show that transcription of EBNA Cp mRNA drops dramatically in the presence of ATO, indicating that the virus has reactivated from the latent to the lytic phase. FIG. 8D graphs the relative transcription of the viral immediate early protein Zta mRNA of LCL<EBV1-13> cells grown three days (“3 D”) in media alone (“control”), media and 45 μM ganciclovir (GCV), media and 10 nM arsenic trioxide (ATO), or media and a combination of these concentrations of GCV and ATO. The results show that the transcription of Zta mRNA has is approximately fourfold higher in cells contacted with ATO, again indicating that the virus has reactivated from the latent to the lytic phase. The expression of EBNA from the Cp promoter verified that these cells exhibit type III latency and are a good model of post transplant lymphoproliferative disease and AIDS associated lymphomas.

### Example 8

This Example sets forth a clinical protocol for demonstrating the efficacy of co-administering the exemplar arsenic compound ATO and the exemplar anti-herpes virus agent ganciclovir.

ATO will be administered at a dose of 0.15 mg/kg/day intravenously over 1 hour once daily, 5 days a week in week 1 and then twice a week in weeks 2 and 3, or alternatively five days a week in weeks 2 and 3, and ganciclovir will be administered intravenously at a dose of 5 mg/kg over 1 hour immediately after administration of the ATO, and again 12 hours later. Marker and non-marker lesions will be assessed at the end of the treatment and the response assigned according to guidelines appropriate for the type of cancer under review. Safety will be assessed by routine physical, laboratory and ECG evaluations. Up to 10 patients will be enrolled into the study. Patients will be followed for 6 months after their last dose of ATO.

### Eligibility Criteria

### Inclusion Criteria

1. Patients must have a histologically or cytologically confirmed EBV+ solid tumor.

2. Patients must have measurable disease, defined as at least one lesion that can be accurately measured in at least one dimension.

3. Patient must have failed or found to be intolerant of standard frontline regimens. There is no limit on the number of prior regimens provided the patient meets all the other eligibility criteria.

4. Adult patients 18 years or older. Because no dosing or adverse event data are currently available on the use of arsenic trioxide in patients <18 years of age, children are excluded from this study but will be eligible for future pediatric single-agent trials, if applicable.

5. Life expectancy of greater than 3 months

6. ECOG performance status of 0, 1 or 2

7. Patients must have normal organ and marrow function as defined below:
absolute neutrophil count >1,500/mL
platelets >100,000/mL
total bilirubin ≦1.5× institutional upper limits of normal
AST(SGOT)/ALT(SGPT) <2.5× institutional upper limit of normal
creatinine ≦1.5× institutional upper limits of normal or creatinine clearance >40 mL/min/1.73 m2 for patients with creatinine levels above institutional normal.
Negative serum pregnancy test within 48 hours before starting study treatment in women with childbearing potential.
Ability to understand and the willingness to sign a written informed consent document.
No history of QTc prolongation syndrome or any other cardiac conduction abnormality evidenced by normal baseline EKG (QTc ≦450 in males and ≦470 in females).
Both men and women and members of all races and ethnic groups are eligible for this trial.

### Exclusion Criteria

1. Need for treatment with chemotherapy (within 4 weeks; 6 weeks for nitrosoureas or mitomycin C); radiotherapy or biologic agents (within 2 weeks) prior to entering the study or those who have not recovered from adverse events due to agents administered more than 4 weeks earlier.

2. Patients may not be receiving any other investigational agents.

3. Patients with uncontrolled symptomatic brain metastases. Patients with no known brain metastasis are not required to undergo screening prior to enrolment.

4. History of allergic reactions attributed to compounds of similar chemical or biologic composition to arsenic trioxide.

5. Uncontrolled intercurrent illness including, but not limited to, ongoing or active infection, symptomatic congestive heart failure, unstable angina pectoris, cardiac arrhythmia, or psychiatric illness/social situations that would limit compliance with study requirements.

6. Pregnant women are excluded from this study because arsenic trioxide is a category D agent with the potential to cause fetal harm. Because there is an unknown but potential risk for adverse events in nursing infants secondary to treatment of the mother with arsenic trioxide, breastfeeding should be discontinued if the mother is treated with arsenic trioxide.

7. HIV-positive patients on combination antiretroviral therapy are ineligible because of the potential for pharmacokinetic interactions with arsenic trioxide. In addition, these patients are at increased risk of lethal infections when treated with marrow-suppressive therapy.

Appropriate studies will be undertaken in patients receiving combination antiretroviral therapy when indicated.

8. Patients who require ongoing treatment with any hematopoietic colony-stimulating growth factors (e.g., G-CSF, GM-CSF) ≦2 weeks prior to starting study drug.

9. Patients who are currently receiving treatment with medication that has the potential to prolong the QT interval or inducing Torsades de Pointes and the treatment cannot either be discontinued or switched to a different medication prior to starting study drug. The following must be discontinued at least 7 days prior to enrollment to be eligible: quinidine, procainamide, disopyramide, amiodarone, sotalol, ibutilide, dofetilide, erythromycins, clarithromycin, chlorpromazine, haloperidol, mesoridazine, thioridazine, pimozide, cisapride, bepridil, droperidol, methadone, chloroquine, domperidone, halofantrine, levomethadyl, pentamidine, sparfloxacin, lidoflazine.

10. Patients who have undergone major surgery ≦2 weeks prior to starting study drug or who have not recovered from side effects of such therapy

11. History of another malignancy within 3 years, except curatively treated basal cell carcinoma of the skin, DCIS, early stage prostate cancer without detectable PSA or excised carcinoma in situ of the cervix.

12. Patient is unable or unwilling to abide by the study protocol

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of reducing the number of Epstein-Ban virus (EBV)-positive cells in a subject diagnosed with an EBV-related condition, said method comprising co-administering effective amounts of an arsenic compound and of an anti-herpes virus antiviral agent, thereby reducing the number of EBV-positive cells in said subject.

2. The method of claim 1, wherein said anti-herpes virus antiviral agent is administered before administration of said arsenic compound.

3. The method of claim 2, wherein said anti-herpes virus antiviral agent is administered two hours or less before administration of said arsenic compound.

4. The method of claim 2, wherein said anti-herpes virus antiviral agent is administered one hour or less before administration of said arsenic compound.

5. The method of claim 1, wherein said anti-herpes virus antiviral agent is administered substantially simultaneously with administration of said arsenic compound.

6. The method of claim 1, wherein said anti-herpes virus antiviral agent is administered within four hours after administration of said arsenic compound.

7. The method of claim 7, wherein said anti-herpes virus antiviral agent is administered approximately two hours after administration of said arsenic compound.

8. The method of claim 7, wherein said anti-herpes virus antiviral agent is administered approximately one hour after administration of said arsenic compound.

9. The method of claim 1, wherein said anti-herpes virus antiviral agent is administered immediately following administration of said arsenic compound.

10. The method of claim 1, wherein said arsenic compound is selected from the group consisting of: an inorganic arsenic compound and an organic arsenic compound.

11. The method of claim 10, wherein said inorganic arsenic compound is arsenic trioxide.

12. The method of claim 1, wherein said arsenic compound is administered intravenously.

13. The method of claim 11, wherein said arsenic trioxide is administered intravenously.

14. The method of claim 13, wherein said arsenic trioxide is administered at a dose of 9 to 45 μg/kg daily.

15. The method of claim 13, wherein said arsenic trioxide is administered at a dose of 15 μg/kg daily.

16. The method of claim 13, wherein said arsenic trioxide is administered at a dose of 0.75 to 5 μg/kg daily.

17. The method of claim 13, wherein said arsenic trioxide is administered at a dose of 1.5 μg/kg daily.

18. The method of claim 1, wherein said anti-herpes virus antiviral agent is selected from the group consisting of: ganciclovir, cidofovir, acyclovir, famciclovir, and valaciclovir.

19. The method of claim 18, wherein said anti-herpes virus antiviral agent is ganciclovir.

20. The method of claim 19, further wherein said ganciclovir is administered intravenously.

21. The method of claim 1, wherein said EBV-related condition is selected from the group consisting of a cancer and EBV-positive idiopathic pulmonary fibrosis.

22. The method of claim 21, wherein said cancer is selected from the group consisting of EBV-positive Hodgkin's lymphoma, Burkitt's lymphoma, nasopharyngeal carcinoma, post-transplant lymphoproliferative disease, AIDS-associated lymphoma, EBV-positive gastric cancer, EBV-positive breast cancer, and EBV-positive lymphoma.

23. The method of claim 1, wherein said subject diagnosed with an EBV-related condition has not been previously been treated with an arsenic compound for, and is not concurrently being treated with an arsenic compound for, acute promyelocytic leukemia.

24. The method of claim 1, provided that if said subject diagnosed with an EBV-related condition has previously or concurrently been diagnosed with a malignant glioma that has been removed by surgery, said subject has not been treated with temozolomide.

25. The method of claim 1, provided that if said subject diagnosed with an EBV-related condition has previously or concurrently been diagnosed with a metastatic endometrial cancer, said subject has not been treated with arsenic.

26. The method of claim 1, provided that said subject diagnosed with an EBV-related condition has not previously or concurrently been diagnosed with small cell lung cancer.

27. The method of claim 1, provided that said subject diagnosed with an EBV-related condition has not previously or concurrently been diagnosed with metastatic melanoma.

28. The method of claim 1, further comprising co-administering a second anti-herpes virus anti-viral agent.

29. A method of reducing the number of cells infected with a herpes virus in a subject in need thereof, said method comprising co-administering effective amounts of an arsenic compound and of an anti-herpes virus antiviral agent, wherein said arsenic compound thereby reactivates latent herpes virus in said infected cells, and said anti-herpes antiviral agent interferes with replication of herpes virus in said infected cells in which said herpes virus has reactivated, thereby reducing the number of cells infected with said herpes virus in said subject.

30. The method of claim 29, wherein said anti-herpes virus antiviral agent is administered two hours or less before administration of said arsenic compound.

31. The method of claim 29, wherein said anti-herpes virus antiviral agent is administered one hour or less before administration of said arsenic compound.

32. The method of claim 29, wherein said anti-herpes virus antiviral agent is administered substantially simultaneously with administration of said arsenic compound.

33. The method of claim 29, wherein said anti-herpes virus antiviral agent is administered immediately after administration of said arsenic compound.

34. The method of claim 29, wherein said anti-herpes virus antiviral agent is administered within one hour after administration of said arsenic compound.

35. The method of claim 29, wherein said arsenic compound is selected from the group consisting of an inorganic arsenic compound and an organic arsenic compound.

36. The method of claim 35, wherein said inorganic arsenic compound is arsenic trioxide.

37. The method of claim 35, wherein said arsenic compound is administered intravenously.

38. The method of claim 36, wherein said arsenic trioxide is administered at a dose of 9 to 45 μg/kg daily.

39. The method of claim 36, wherein said arsenic trioxide is administered at a dose of 15 μg/kg daily.

40. The method of claim 36, wherein said arsenic trioxide is administered at a dose of 0.75 to 5 μg/kg daily.

41. The method of claim 36, wherein said arsenic trioxide is administered at a dose of 1.5 μg/kg daily.

42. The method of claim 29, wherein said subject in need thereof has not previously been treated with chemotherapy or radiation for a cancer.

43. The method of claim 29, wherein said subject in need thereof has not been previously or concurrently been treated for with acute promyelocytic leukemia.

44. The method of claim 29, further comprising a second anti-herpes virus anti-viral agent.

45. The method of claim 29, wherein said herpes virus is varicella zoster.

46. The method of claim 29, wherein said herpes virus is herpes simplex type 2.
